(19) United States
(12) Patent Application Publication
(10) Pub. No.: US 2004/0072356 A1
(43) Pub. Date: Apr. 15, 2004

(54) Methods and apparatuses for characterizing stability of biological molecules

(76) Inventors: Guillermo Senisterra, Hamilton (CA); Eugene Markin, Kitchener (CA); Ken Yamazaki, Toronto (CA); Raymond Hui, Toronto (CA)

Correspondence Address: Jennifer K. Holmes, Foley Hoag LLP, 155 Seaport Boulevard, Boston, MA, 02210, US

(21) Appl. No.: 10/370,268

(22) Filed: Feb. 20, 2003

Related U.S. Application Data
(60) Provisional application No. 60/358,190, filed on Feb. 20, 2002.

(51) Int. Cl. G01N 33/48

(52) U.S. Cl. 436/63; 436/172; 422/73; 422/82.08

(57) ABSTRACT

The invention provides methods and apparatuses for characterizing the folding and aggregation dynamics of biological molecules, including stability of biological molecules. The methods and apparatuses of the invention can be used, for example, to identify conditions that affect the stability of a biological molecule, to identify compounds or ligands that bind to a biological molecule, and to identify compounds that modulate the interaction between a biological molecule and a ligand.

### RELATED APPLICATIONS

This application claims the benefit of priority to U.S. Provisional Patent Application No. 60/358,190, filed on Feb. 20, 2002, which application is hereby incorporated by reference in its entirety.

### BACKGROUND OF THE INVENTION

Recent advances in genomics research provide an opportunity for rapid progress in the identification of novel drug targets. The complete genomic sequences for a number of microorganisms are already available. However, knowledge of the complete genomic sequence is only the first step in a long process toward discovery of a viable drug target. Targeted approaches to drug discovery may involve a variety of steps including annotation of the genomic sequence to identify open reading frames (ORFs), determination of the essentiality of the protein encoded by the ORF, and determination of the mechanism of action of the gene product. In addition to increasing the speed with which novel drug targets are identified, it is also important to make parallel advances in screening the potential drug targets in order to identify drugs which modulate the function of the target.

New technologies are required to facilitate the transition from gene sequence (or genomics) to gene function (or functional genomics). Classification of proteins of unknown function based on nucleotide or amino acid homology with proteins of known function may be difficult. While conservation between amino acid sequences generally indicates a conservation of structure and function, specific changes at key residues can lead to significant variation in the biochemical, biophysical, and/or functional properties of a protein.

To facilitate the study of proteins, it is important to have the proteins available in a reasonably stable form. In addition, characterization of proteins and identification of drugs requires the identification of molecules that interact with the proteins. Therefore, methods for identifying conditions that stabilize proteins and methods for identifying molecules that bind to the proteins are highly desirable.

Typical screening techniques are target-specific. In other words, it is necessary to develop custom assays for a given target which is extremely time-consuming. Furthermore, existing non-specific screening techniques do not provide sufficiently rich data and typically require additional screening using target-specific techniques or labeling of targets (e.g. with fluorescent probes).

In structural proteomics, x-ray crystallography is a powerful technique for solving the three-dimensional structure of a protein. A key step in this technique is protein crystallization. Increasingly researchers are interested in setting crystal screens at progressively higher rates, thus demanding an effective and efficient means for pre-selecting conditions favorable to crystallization. Therefore, new and improved methods for pre-selection of conditions, such as characterization of proteins using biophysical and biochemical means, are highly desirable.

Outside of drug discovery, high throughput experimentation is becoming more commonplace as a discovery tool. For example, combinatorial chemistry, a high throughput technique common in drug discovery, is emerging as an important tool in material discovery in the chemical and electronics industries. The demand for high throughput discovery in all of the above areas of applications requires high throughput detection, including real-time monitoring, and screening for desired properties.

Light scattering is recognized in the art as an effective and sensitive means for detection and characterization of small particles, particularly those in solution. In applications such as those mentioned above where formation of small particles naturally takes place, process status can be monitored by means of light scattering. However, commercially available light scattering instruments can measure only one sample at a time and are typically expensive. To use a number of such instruments to monitor a multiplicity of samples renders the cost prohibitive. Therefore, there is a great need for methods and apparatus that permit monitoring of light scattering of a multiplicity of samples essentially simultaneously.

### SUMMARY OF THE INVENTION

In one aspect, the invention provides, a method for characterizing aggregation of a plurality of biological samples, comprising:

a) providing a plurality of biological samples, wherein each composition comprises at least one biological molecule;

b) exposing the plurality of biological samples to one or more light sources; and

c) determining the amount of light scattered by said plurality of biological samples upon exposure to said one or more light sources, thereby characterizing aggregation of said biological samples.

In one embodiment, the light source may be one or more lasers. For example, the plurality of biological samples may be exposed to a plurality of lasers or the beam of one or more lasers may be split in order to expose the plurality of biological samples to the laser light essentially simultaneously. In an alternative embodiment, the light source may be one or more non-laser lights, such as for example, a light emitting diode (LED), a white light source, a monochromatic light source, an incandescent light source, a Xenon-arc lamp, a tungsten-halogen lamp, an ultraviolet light source, a luminescent light source, and a low intensity light source having an intensity in a range of 1.5 to 2.0 μW/mm². In an exemplary embodiment, the non-laser light is a plurality of light emitting diodes (LEDs). In various embodiments, determining the amount of light scattered may comprise detecting the amount of non-scattered light, detecting the amount of scattered light, or both.

In another embodiment, the light source may be passed through an optical filter, such as, for example, a monochromator or a polarizing filter, before exposure to the plurality of biological samples.

In various embodiments, the plurality of biological samples comprises at least about 2, 3, 4, 5, 10, 15, 20, 50, 100, 200, 500, 100, or more biological samples. In exemplary embodiments, the plurality of biological samples comprises at least about 96, 384, 1536 biological samples, for example, as available in various configurations of standard microtiter plates. In another embodiment, the plurality of biological samples are contained in a plurality of wells of a microtiter plate.

In another embodiment, one or more biological samples comprises at least one biological molecules, such as, for example, a polynucleotide or a polypeptide. In another embodiment, one or more biological samples comprises a mixture of biological molecules, such as, for example, a mixture of polypeptides, a mixture of polynucleotides, or a mixture of polypeptides and polynucleotides. In other embodiments, the plurality of samples may comprise at least one biological molecule in a plurality of test conditions, at least one mixture of biological molecules in a plurality of test conditions, a plurality of biological molecule in one or more test conditions, a plurality of biological molecule in a plurality of test conditions, etc.

In another embodiment, the methods as described herein may further comprise determining the aggregation rate (k_{agg}) of said one or more biological samples. In other embodiments, characterizing aggregation may comprise determining one or more of the following: the aggregation state of the biological sample, the aggregation kinetics of the biological sample, or the aggregation dynamics of the biological sample. In other embodiments, the methods comprise characterizing aggregation of said plurality of biological samples as a function of time and/or temperature.

In another embodiment, the methods as described herein may comprise preparing the plurality of compositions in an automated fashion.

In another embodiment, the methods described herein comprise comparing a property of aggregation of at least one biological sample in at least one test condition to a property of aggregation of said biological sample in a reference condition. A property of aggregation for at least one biological sample may be determined, for example, in at least about 2, 5, 10, 20, 50, 100, or more test conditions. In an exemplary embodiment, a property of aggregation for a plurality of biological samples is determined in a plurality of test conditions. Exemplary test conditions, include, for example, differences as compared to a reference condition in one or more of the following: a biochemical condition, pressure, electric current, time, concentration of the biological molecule, and presence of a test compound. Exemplary, biochemical conditions, include, for example, pH, ionic strength, salt concentration, oxidizing agent, reducing agent, detergent, glycerol, metal ions, salt, cofactor concentration, ligand concentration, and/or coenzyme concentration. In an exemplary embodiment, a test condition comprises the presence of one or more potential ligands of a biological molecule in a biological sample.

In another embodiment, the methods described herein may further comprise bringing the temperature of said plurality of biological samples to one or more end temperatures before determining the amount of light scattered. In one embodiment, characterizing aggregation of at least one biological sample may be determined at one or more end temperatures, optionally, as a function of time. In another embodiment, characterizing aggregation of a plurality of biological samples may be determined over a range of end temperatures. In an exemplary embodiment, characterizing aggregation of at least one biological sample may be determined over a range of end temperatures by essentially simultaneously bringing a plurality of biological samples comprising a biological molecule to a plurality of end temperatures. In another exemplary embodiment, characterizing aggregation of at least one biological sample may be determined over a range of end temperatures by sequentially bringing a biological sample to a plurality of end temperatures. In various embodiments, the range of end temperatures may be sequentially increased over time. In certain embodiments, characterizing aggregation of at least one biological sample may be determined, for example, at about 2, 5, 10, 20, 50, or more end temperatures. In another embodiment, a plurality of biological samples may be exposed to a temperature gradient to allow characterizing aggregation of said plurality of biological samples as a function of temperature.

In various embodiments of the invention, the extent of unfolding of one or more biological molecules in a biological sample may be determined in addition to characterizing aggregation of one or more biological molecules in the biological sample. The extent of unfolding a biological molecule may be determined, for example, by fluorescence emission, circular dichroism, or differential scanning calorimetry. In certain embodiments, the invention further comprises determining the rate of unfolding (kᵤ) and the rate of aggregation (k_{agg}) of one or more biological molecules in one or more biological samples. In another embodiment, the methods described herein may further comprise determining the temperature of unfolding (Tₘ) of said one or more biological molecules.

In another embodiment, the invention provides methods for predicting optimal conditions for crystallization, purification, folding, and/or refolding; high throughput screening of target molecules; high throughput study of kinetics and/or dynamics of unfolding; kinetics and/or dynamics of aggregation; kinetics and/or dynamics of both unfolding and aggregation; dynamics of folding; and biophysical characterization of biological samples. In exemplary embodiments, such methods involve characterizing biological samples under a variety of physical and biochemical conditions, and determining, for example, molecular configuration and conformation, solubility, structural stability, etc.

In another embodiment, the invention provides methods for identifying a condition in which a biological molecule has a different stability relative to its stability in a reference condition or relative to other conditions under study. In an exemplary embodiment, the invention comprises (a) providing a composition comprising a biological molecule in a test condition; (b) bringing the temperature of the composition to an end temperature; (c) determining the extent of aggregation of the biological molecule in the composition as a function of time over a period extending past the time point at which the temperature of the solution attains the end temperature; (d) obtaining a characteristic of aggregation of the biological molecule in the test solution from the extent of aggregation obtained as a function of time in (c); and (e) comparing the characteristic of aggregation obtained in (d) with the characteristic of aggregation of the biomolecule in the reference condition, wherein a different characteristic of aggregation of the biological molecule in the test condition relative to the reference condition indicates that the test condition is a condition in which the biological molecule has a different stability relative to its stability in the reference condition. In various embodiments, the end temperature may be lower, lower than, or substantially equivalent to, the aggregation temperature of the biological molecule in the reference condition. The method can be used to identify conditions in which a biological molecule has a higher stability relative to its stability in a reference condition and to compare relative efficacies of different conditions in stabilizing the biological molecule. The biological molecule can be a protein. In an exemplary embodiment, determining the extent of aggregation of the biological molecule in the composition as a function of time is conducted essentially only when the temperature of the composition is at the end temperature. The characteristic of aggregation may be the rate of unfolding (k) or the rate of aggregation (k_{agg}), respectively. The method may comprise first determining the aggregation temperature of the biological molecule in the reference solution. In certain embodiments, the temperature of step (b) can be lower than the aggregation temperature of the biological molecule in the reference solution by at least about 5° C.

The test condition can differ from the reference condition in one or more of the following: a biochemical condition, pressure, electric current, time, concentration of the biological molecule, and presence of a test compound. The test condition can differ from the reference condition in a biochemical condition selected from the group consisting of pH, ionic strength, salt concentration, oxidizing agent, reducing agent, detergent, glycerol, metal ions, salt, cofactor concentration, ligand concentration and coenzyme concentration. The test condition can comprise a potential ligand of the biological molecule not known to bind to the biological molecule, and wherein a lower k_{agg} of the biological molecule in the test condition relative to the reference condition indicates that the potential ligand interacts with the biological molecule.

The method can comprise determining the extent of unfolding of the biological molecule by fluorescence emission, e.g., with 4,4′-dianilino-1,1-binaphthyl-5,5-disulfonic acid (bis-ANS). The method can comprise determining the extent of aggregation of the biological molecule by measuring absorption of ultraviolet light, absorption of visible light, changes in turbidity, or changes in the polar properties of light.

The method may further comprise increasing the temperature of the composition after step (c) and repeating steps (c) to (e) at the higher temperature. In another embodiment, the composition forms a temperature gradient and the method comprises determining the extent of aggregation of the biological molecule in at least two locations of the gradient.

The invention also provides a method for identifying a condition in a plurality of conditions in which a biological molecule has a higher stability relative to its stability in the other conditions, comprising (a) providing a plurality of compositions each comprising essentially the same biological molecule in a plurality of different conditions; (b) changing the temperature of the composition; (c) determining the extent of aggregation of the biological molecule in the compositions as a function of time over a period extending past the time point at which the temperature was changed; (d) obtaining the k_{aggs} of the biological molecule in the test conditions from the extent of aggregation obtained as a function of time in (c); and (e) comparing the k_{aggs} obtained in (d) with each other, respectively, wherein the test condition in which the k_{agg} is the highest among the plurality of test conditions is a condition in which the stability of the biological molecule is higher relative to its stability in the other test conditions. Step (e) may further comprise comparing the k_{aggs} obtained in (d) with the k_{agg}, respectively, of the biological molecule in the reference condition. The plurality of test conditions can comprise at least 5, 10, or 100 test conditions. The plurality of compositions can be in a plurality of wells of a microwell plate and the method can be conducted in an automated manner. In one embodiment, step (b) may involve bringing the temperature of the compositions to a temperature that is slightly lower than the aggregation temperature of the biological molecule in the reference condition.

In one embodiment, the invention provides a method for identifying a condition in which a biological molecule has a different stability relative to its stability in a reference condition, comprising: (a) providing a composition comprising a biological molecule in a test condition; (b) increasing the temperature of the composition over time; (c) determining the extent of unfolding and aggregation of the biological molecule in an essentially simultaneous manner during the increase in temperature; (d) obtaining a characteristic of unfolding and aggregation of the biological molecule in the test condition from the extent of unfolding and aggregation obtained in (c); and (e) comparing the characteristic of unfolding and aggregation obtained in (d) with that of the biological molecule in the reference condition; wherein a different characteristic of aggregation of the biological molecule in the test condition relative to the reference condition indicates that the test condition is a condition in which the biological molecule has a different stability relative to its stability in the reference condition. The characteristic of unfolding and aggregation can be the temperature of unfolding (Tₘ) and the temperature of aggregation (T_{agg}), respectively. The extent of unfolding can be determined by bis-ANS fluorescence and the extent of aggregation can be determined by light scattering. The composition can be alternatively exposed a UV light and a light source for light scattering during the increase in temperature. The UV light and light source for light scattering can be computer controlled to be switched on and off alternatively for fluorescence and light scattering, respectively. In exemplary embodiments, a light source for light scattering may be one or more of the following: a laser, a light emitting diode (LED), a cluster of LEDs, a white light source, a monochromatic light source, an incandescent light source, a Xenon-arc lamp, a tungsten-halogen lamp, an ultraviolet light source, a luminescent light source, and/or a low intensity light source with an intensity in a range of 1.5 to 2.0 μW/mm².

In another embodiment, the invention provides methods for identifying a condition among a plurality of conditions in which a biological molecule has a higher stability relative to its stability in the other conditions, comprising: (a) providing a plurality of compositions comprising essentially the same biological molecule in a plurality of different test conditions; (b) increasing the temperature of the plurality of compositions over time; (c) determining the extent of unfolding and aggregation of the biological molecule in the plurality of compositions in an essentially simultaneous manner during the increase in temperature; (d) obtaining the Tₘ and T_{agg} of the biological molecule in each of the test conditions from the extent of unfolding and aggregation obtained in (c); and (e) comparing the Tₘₛ and T_{aggs} obtained in (d) with one another, wherein the test condition in which the kᵤ or k_{agg} is the lowest among the plurality of test conditions is a condition in which the stability of the biological molecule is higher relative to its stability in the other test conditions. The temperature of the plurality of compositions can be increased essentially simultaneously over time.

Also within the scope of the invention are computer readable media and databases comprising the results of the methods of the invention. Kits and apparatuses are also provided.

The invention further provides an apparatus for measuring an extent of aggregation in at least one molecular sample. In an exemplary embodiment, the apparatus comprises a light source positioned to illuminate the molecular sample; a sample container for containing the molecular sample; a light guide positioned in an optical path between the light source and the sample container to direct light from the light source into the sample container; a scattered light detector positioned to receive the light passing through the molecular sample and scattered from the molecular sample at an angle from the optical path of the light entering the sample from the light guide, the scattered light detector producing a signal proportional to the received scattered light; and a processor in communication with the scattered light detector to receive and process the signal from the scattered light detector to determine the extent of aggregation in the at least one molecular sample.

In one embodiment, the light guide is positioned at an angle with respect to an optical path between the at least one molecular sample and the detector, such that the angle is less than 45° and preferably in a range from 15° to 30°.

In one embodiment, the detector can be one of a photomultiplier and a charged-couple device (CCD). The apparatus can include a luminescence detector positioned to receive fluorescence emanating from the at least one molecular sample, the luminescence detector producing a signal proportional to the received fluorescence, the processor receiving and processing the signal from the luminescence detector to determine an extent of unfolding in the molecular sample. A switch can select which detector the processor can receive the signal from.

In one embodiment, the apparatus includes a luminescent light source to illuminate the molecular sample. The detector can receive fluorescence emanating from the illuminated sample resulting from the illumination by the luminescent light source and can produce a signal proportional to the received fluorescence. The processor can determine an extent of unfolding in the molecular sample based on the signal received from the detector. A switch can selectively operate the luminescent light source.

In one embodiment the sample container includes an array of sample wells, each sample well being sized to contain one of the molecular samples. The sample wells can be spatially separated from each other to inhibit cross-contamination and can be optically isolated to inhibit scattered light from one sample from illuminating other samples. The light guide can be a collimator positioned in the optical path between the light source and the sample container to substantially collimate light from the light source into the sample wells. The collimator can be an array of optical fibers, wherein the optical fibers are each optically aligned with a respective sample well within the sample container.

In one embodiment, the apparatus can include means for selectively directing and/or occluding light from the light source to at least one of the sample wells. In one embodiment, the apparatus can include means for selectively directing or occluding scattered light from at least one of the sample wells to the scattered light detector.

In one embodiment, the apparatus can include a heating element for heating the sample container. The heating element can be configured to create a temperature gradient across the sample container and can be configured to selectively heat at least one selected sample well, such that the at least one selected sample well is heated to a temperature distinct from other sample wells.

The light source can be one of a number of sources, including, for example, a laser, a light emitting diode (LED), a cluster of LEDs, a white light source, a monochromatic light source, an incandescent light source, a Xenon-arc lamp, a tungsten-halogen lamp, an ultraviolet light source and a luminescent light source. The light source can be a low intensity light source having an intensity in a range of 1.5 to 2.0 μW/mm². A monochromator can be positioned in the optical path between the light source and the molecular sample to illuminate the molecular sample with monochromatic light.

In one embodiment, an apparatus measures an extent of aggregation in a plurality of molecular samples. The apparatus can include a sample container for containing the molecular samples; a light source positioned to illuminate selected ones of the molecular samples; a scattered light detector positioned to receive the light passing through the selected ones of the molecular samples and scattered from the selected ones of the molecular samples, the scattered light detector producing a signal proportional to the received scattered light; and a processor in communication with the scattered light detector to receive and process the signal from the scattered light detector to determine the extent of aggregation in the selected ones of the molecular samples.

The apparatus can include a collimator positioned in an optical path between the light source and the sample container to substantially collimate light from the light source into the molecular samples. The collimator can be an array of optical fibers that can each be optically aligned with a respective molecular sample within the sample container. The collimator can be positioned at an angle with respect to an optical path between the molecular samples and the detector, wherein the angle is less that 45° and preferably in a range from 15° to 30°.

The sample container can include an array of sample wells, each sample well being sized to contain one of the molecular samples and each sample well being optically isolated from other sample wells of the array to inhibit scattered light from the molecular sample in the sample well from illuminating the molecular sample in the other sample wells. The apparatus can further include optical directing means for selectively directing light from the light source to at least one of the molecular samples, wherein the optical directing means can include micro-electromechanical devices selectively controlling movements of an array of directing optics to form an optical path between the light source and the at least one molecular sample.

Means for selectively occluding the optical path between the light source and the molecular samples, means for selectively directing scattered light from the molecular samples to the scattered light detector, or means for selectively occluding light from the molecular samples to the scattered light detector can be provided. The light source can be one of a laser, a light emitting diode (LED), a cluster of LEDs, a white light source, a monochromatic light source, an incandescent light source, a Xenon-arc lamp, a tungsten-halogen lamp, an ultraviolet light source, or a luminescent light source. The light source can be a low intensity light source with an intensity in a range of 1.5 to 2.0 μW/mm².

An apparatus for measuring an extent of aggregation in a plurality of molecular samples can include an array of sample wells, each sample well being sized to contain one of the molecular samples; a light source positioned to illuminate selected ones of the sample wells; a light guide positioned in an optical path between the light source and the sample container to direct light from the light source into the sample wells; a light detector positioned to receive at least one of scattered light and fluorescence from the molecular samples in the selected ones of the sample wells, the light detector producing a signal proportional to the received light; and a processor in communication with the light detector to receive and process the signal from the light detector to determine the extent of aggregation in the molecular samples in the selected ones of the sample wells when the received light is scattered light and to determine the extent of unfolding in the molecular samples in the selected ones of the sample wells when the received light is fluorescence.

In one embodiment, the light source can include a low intensity light source and/or a luminescent light source. The light emitted from the low intensity light source can pass through the selected ones of the sample wells and be scattered by the molecular sample to be received as scattered light at the detector. The detector can receive fluorescence emanating from the molecular samples in the selected ones of the sample wells that have been illuminated by the luminescent light source. A switch can selectively operate the low intensity light source and the luminescent light source.

In one embodiment, the detector can include a scattered light detector to receive light from the light source passing through the selected ones of the sample wells and scattered by the molecular samples and a fluorescence detector to receive fluorescence emanating from the molecular samples in the selected ones of the sample wells illuminated by the light source. A switch can be operated to select between the processor receiving the signal from scattered light detector and the processor receiving the signal from the luminescence detector.

An apparatus for measuring an extent of aggregation in a plurality of molecular samples and/or an extent of unfolding in a plurality of molecular samples can include an array of sample wells, each sample well being sized to contain one of the molecular samples; a first light source positioned to illuminate selected ones of the sample wells; a second light source positioned to illuminate the same, or other selected ones of the sample wells; a light guide positioned in an optical path between the light sources and the sample container to direct light from the light sources into the sample wells; a light detector positioned to receive light from the first light source passing through the selected ones of the sample wells and scattered by the molecular sample and to receive fluorescence emanating from the molecular samples in the selected ones of the sample wells being illuminated by the second light source, the light detector producing a signal proportional to the received light; and a processor in communication with the light detector to receive and process the signal from the light detector to determine the extent of aggregation in the molecular samples in the first selected ones of the sample wells when the received light is scattered light and to determine the extent of unfolding in the molecular samples in the second selected ones of the sample wells when the received light is fluorescence.

In one embodiment, the first light source can be a low intensity light source. A switch can be included such that the first light source and the second light can be selectively operated. The detector can include a scattered light detector and a fluorescence detector. A switch can be included to select between the processor receiving the signal from the scattered light detector and the processor receiving the signal from the fluorescence detector.

### BRIEF DESCRIPTION OF THE FIGURES

FIG. 1 shows an illustrative isometric view of an apparatus for measuring aggregation in molecular samples.

FIG. 2 shows an illustrative cross-sectional view taken along the line 2-2 in FIG. 1.

FIG. 3 shows an illustrative cross-section view, corresponding to that of FIG. 2, of an alternative embodiment of the apparatus of FIG. 1.

FIG. 4 illustrates types of scattering by macromolecules.

FIGS. 5A and B show the light scattering and fluorescence of a protein as a function of time.

FIG. 6 shows the fluoresence of the protein as a function of time at 55° C.

FIG. 7 shows an example of a selective illumination pattern which minimizes or inhibits cross-talk.

### DETAILED DESCRIPTION OF THE INVENTION

As used herein, the following terms and phrases shall have the meanings set forth below. Unless defined otherwise, all technical and scientific terms used herein have the same meaning as commonly understood to one of ordinary skill in the art to which this invention belongs.

The singular forms “a,” “an,” and “the” include plural reference unless the context clearly dictates otherwise.

The term “biochemical conditions” encompasses any characteristic of a physical, chemical, or biochemical process or reaction. In exemplary embodiments, the term refers to conditions including, for example, temperature, pressure, protein concentration, pH, ionic strength, salt concentration, time, electric current, potential difference, concentrations of cofactor, coenzyme, oxidizing agents, reducing agents, detergents, metal ion, ligands, or glycerol.

The term “biophysical characteristics” refer to physical characteristics of a biological molecule relevant to the biological function of the molecule, including its state, solubility, structure, etc. The term “biophysical characterization” refers to tests or processes carried out to determine a sample's biophysical characteristics.

The term “carrier” encompasses a platform or other object, of any shape, which itself is capable of supporting at least two containers. The carrier can be made of any material, including, but not limited to, glass, plastic, or metal. Preferably, the carrier is a multiwell microplate. The terms microplate and microtiter plate are synonymous. The carrier can be removed from the heating element. Each carrier can hold a plurality of containers.

The term “combinatorial library” refers to a plurality of molecules or compounds which are formed by combining, in close to every possible way for a given compound length, a set of chemical or biochemical building blocks which may or may not be related in structure. Alternatively, the term can refer to a plurality of chemical or biochemical compounds which are formed by selectively combining a particular set of chemical building blocks. Combinatorial libraries can be constructed according to methods familiar to those skilled in the art. For example, see Rapoport et al., Immunology Today 16:4349 (1995); Sepetov, N. F. et al., Proc. Natl. Acad. Sci. U.S.A. 92:5426-5430 (1995); Gallop, M. A. et al., J. Med. Chem. 9:1233-1251 (1994); Gordon, E. M. et al., J. Med. Chem. 37:1385-1401 (1994); Stankova, M. et al., Peptide Res. 7:292-298 (1994); Erb, E. et al., Proc. Natl. Acad. Sci. U.S.A. 91:11422-11426 (1994); DeWitt, S. H. et al., Proc. Natl. Acad. Sci. U.S.A. 90:6909-6913 (1993); Barbas, C. F. et al., Proc. Natl. Acad. Sci. U.S.A. 89:4457-4461 (1992); Brenner, S. et al. Proc. Natl. Acad. Sci. U.S.A. 89:5381-5383 (1992); Lam, K. S. et al., Nature 354:82-84 (1991); Devlin, J. J. et al., Science 245:404-406 (1990); Cwirla, S. E. et al., Proc. Natl. Acad. Sci. U.S.A. 87:6378-6382 (1990); Scott, J. K. et al., Science 249:386-390 (1990). In an exemplary embodiment, the term “combinatorial library” refers to a diversity chemical library, as set forth in U.S. Pat. No. 5,463,564. Regardless of the manner in which a combinatorial library is constructed, substantially every different molecule or compound in the library is catalogued for future reference.

The term “compound library” refers to a plurality of molecules or compounds which were not formed using the combinatorial approach of combining chemical or biochemical building blocks. Instead, a compound library is a plurality of molecules or compounds which are accumulated and are stored for use in binding assays, such as, for example, binding assays between a target molecule and a ligand. Substantially every different molecule or compound in the compound library is catalogued for future reference.

The term “container” refers to any vessel or chamber, in which the receptor and molecule to be tested for binding can be placed. The term “container” encompasses reaction tubes (e.g., test tubes, microtubes, vials, etc.). In an exemplary embodiment, the term “container” refers to one or more wells in a multiwell microplate or microtiter plate. The term “sample” refers to the contents of a container.

As used herein, the “folded state” of a protein refers to the native or undenatured form of the protein as it is present under physiological conditions, with secondary, tertiary and/or quaternary structures intact. Physiological conditions include conditions similar to the natural environment of the protein, or conditions under which it is stable after expression, isolation, and/or purification, i.e. before exposure to denaturing conditions. Similarly, the “unfolded state” refers to a situation in which the polypeptide has lost elements of its secondary, tertiary and/or quaternary structure that are present in its “folded state.” It will be recognized by those skilled in the art that it is difficult to determine experimentally when a polypeptide has become completely unfolded (i.e., when a polypeptide has lost all elements of secondary, tertiary, and/or quaternary structure). Thus, the term “unfolded state” as used herein encompasses partial or total unfolding.

The term “capable of denaturing” refers to the ability to cause the loss of secondary, tertiary, and/or quaternary structure through unfolding, uncoiling, or untwisting.

The terms “folding,” “refolding,” and “renaturing” refer to the acquisition of the correct secondary, tertiary, and/or quaternary structure, of a protein or a nucleic acid, which affords a full chemical and/or biological function of the biomolecule.

The term “aggregation” refers to the association of two or more biological molecules. In one embodiment, the term is meant to encompass crystallization, native aggregation, and/or pathological aggregation. “Crystallization” refers to the aggregation of molecules in an orderly fashion such that essentially all molecules are oriented in essentially the same way. “Native aggregation” refers to the formation of homo- or hetero- dimers, trimers, etc., of biological molecules, especially proteins, which interact to form a multimeric molecule having a biological function. “Pathological aggregation” refers to the association of two or more biological molecules due to hydrophobic interactions. Pathological aggregates of proteins often are not biologically active. In an exemplary embodiment, the term “aggregation” refers to pathological aggregation of biological molecules and excludes crystallization and native aggregation.

The term “characterizing aggregation”, with reference to a biological sample, refers to determining a property of aggregation of one or more biological molecules in the biological sample. The term “property of aggregation” is meant to encompass the extent of aggregation, aggregation state, aggregation kinetics, and/or aggregation dynamics of a biological molecule.

The term “extent of aggregation”, with reference to a biological molecule, refers to the proportion by mass of biological molecules in a biological sample that are in aggregates relative to the total mass of the biological molecules in the sample under a given set of conditions. The extent of aggregation can be determined by a variety of methods, such as those described herein.

“Extent of unfolding” or “extent of denaturation” of a biological molecule refers to the extent of unfolding of the biological molecule, i.e., the extent of changes in its secondary, tertiary and/or quaternary structure. Extent of unfolding of a biological molecule also refers to the proportion of biological molecules in a composition that are partially or completely unfolded relative to those that are in their native configuration under particular conditions. The extent of unfolding can be determined by a variety of methods, such as those described herein.

“Characteristics of unfolding” or “characteristics of aggregation” refer to parameters, or changes in parameters, that reflect the extent of unfolding or aggregation of a sample, respectively. Characteristics of unfolding or aggregation include, for example, thermal unfolding or aggregation curves, or portions thereof, e.g., Tₘ or T_{agg}—the transition temperatures from the unfolding or aggregation curves respectively, and rates of unfolding (kᵤ) and aggregation (k_{agg}).

A “thermal unfolding curve” is a plot of the physical change associated with the unfolding of a protein or a nucleic acid as a function of temperature. See, for example, Davidson et al, Nature Structure Biology 2:859 (1995); Clegg, R. M. et al., Proc. Natl. Acad. Sci. U.S.A. 90:2994-2998 (1993). The “midpoint temperature” or “Tₘ” is the temperature on a thermal unfolding curve at which the ratio of folded vs. unfolded protein is 1:1. It is also referred to as “transition temperature” or “melting temperature”. The Tₘ can be readily determined using methods well known to those skilled in the art. See, for example, Weber, P. C. et al., J. Am. Chem. Soc. 116:2717-2724 (1994); Clegg, R. M. et al., Proc. Nati. Acad. Sci. U.S.A. 90:2994-2998 (1993).

A “thermal aggregation curve” is a plot of physical change associated with the aggregation of a biological molecule as a function of temperature. The “aggregation transition temperature”, or T_{agg}, is the temperature on the thermal aggregation curve at which the ratio of aggregated vs. unaggregated protein is 1:1. It is also referred to as the “aggregation temperature”.

The term “fluorescence probe molecule” refers to a fluorophore, which is a molecule or a compound capable of binding to an unfolded or denatured receptor and, after excitement by light of a defined wavelength, emits fluorescent energy. The term fluorescence probe molecule encompasses all fluorophores. More specifically, for proteins, the term encompasses fluorophores such as thioinosine, and N-ethenoadenosine, formycin, dansyl derivatives, fluorescein derivatives, 6-propionyl-2-(dimethylamino)-napthalene (PRODAN), 2-anilinonapthalene, and N-arylamino-naphthalene sulfonate derivatives such as 1-anilinonaphthalene-8-sulfonate (1,8-ANS), 2-anilinonaphthalene-6-sulfonate (2,6-ANS), 2-aminonaphthalene-6-sulfonate, N,N-imethyl-2-aminonaphthalene-6-sulfonate, N-phenyl-2-aminonaphthalene, N-cyclohexyl-2-aminonaphthalene-6-sulfonate, N-phenyl-2-aminonaphthalene-6-sulfonate, N-phenyl-N-methyl-2-aminonaphthalene-6-sulfonate, N-(o-toluyl)-2-aminonaphthalene-6-sulfonate, N-(m-toluyl)-2-aminonaphthalene-6-sulfonate, N-(p-toluyl)-2-aminonaphthalene-6-sulfonate, 2-(p-toluidinyl)-naphthalene-6-sulfonic acid (2,6-TNS), 4-(dicyanovinyl) julolidine (DCVJ), 6-dodecanoyl-2-dimethylaminonaphthalene (LAURDAN), 6-hexadecanoyl-2-(((2-(trimethylammonium)ethyl)methyl)amino)naphthalenechloride (PATMAN), nile red, N-phenyl-1-naphthylamine, 1,1-dicyano-2-[6-(dimethylamino) naphthalen-2-yl]propene (DDNP), 4,4′-dianilino-1,1-binaphthyl-5,5-disulfonic acid (bis-ANS), and DAPOXYL™ derivatives (Molecular Probes, Eugene, Oreg.). In an exemplary embodiment, the term refers to 1,8-ANS or 2,6-TNS in association with proteins. A “donor fluorophore” is one which, when excited by light, will emit fluorescent energy. The energy emitted by the donor fluorophore is absorbed by the acceptor fluorophore. The term “donor fluorophore” encompasses all fluorophores including, but not limited to, carboxyfluorescein, iodoacetamidofluorescein, and fluorescein isothiocyanate. The term “acceptor fluorophore” encompasses all fluorophores including, but not limited to, iodoacetamidoeosin and tetramethylrhodamine.

The term “polypeptide”, and the terms “protein” and “peptide” which are used interchangeably herein, refers to a polymer of amino acids. Exemplary polypeptides include gene products, naturally-occurring proteins, recombinant polypeptides, fragments, and other equivalents, variants and analogs of the foregoing. In certain instances, a protein may comprise two or more polypeptide chains that are associated through covalent or non-covalent interactions.

The terms “recombinant protein” or “recombinant polypeptide” refer to a polypeptide which is produced by recombinant DNA techniques. An example of such techniques includes the case when DNA encoding the expressed protein is inserted into a suitable expression vector which is in turn used to transform a host cell to produce the protein or polypeptide encoded by the DNA.

The term “target molecule” encompasses peptides, proteins, nucleic acids, and other biological molecules. The term encompasses both enzymes and proteins which are not enzymes. The term encompasses monomeric and multimeric proteins. Multimeric proteins may be homomeric or heteromeric. The term encompasses nucleic acids comprising at least two nucleotides, such as oligonucleotides. Nucleic acids can be single-stranded, double-stranded or triple-stranded. The term encompasses a nucleic acid which is a synthetic oligonucleotide, a portion of a recombinant DNA molecule, or a portion of chromosomal DNA. The term target molecule also encompasses portions of peptides, proteins, and other receptors which are capable of acquiring secondary, tertiary, or quaternary structure through folding, coiling or twisting. The target molecule may be substituted with substituents including, but not limited to, cofactors, coenzymes, prosthetic groups, lipids, oligosaccharides, or phosphate groups.

As used herein, the term “target protein” refers to a test molecule which may be a peptide, protein or protein complex for which characterization of the stability and/or identification of a ligand or binding partner is desired. Target proteins include without limitation peptides or proteins known or believed to be involved in the etiology of a given disease, condition or pathophysiological state, or in the regulation of physiological function. Target proteins may be derived from any living organism, such as a prokaryotes, virus, and eukaryotes, including, for example, vertebrates, particularly mammals, and even more particularly humans. For use in the present invention, it is not necessary that the protein's biochemical function be specifically identified. Target proteins include without limitation receptors, enzymes, oncogene products, tumor suppressor gene products, vital proteins, and transcription factors, either in purified form or as part of a complex mixture of proteins and other compounds. Furthermore, target proteins may comprise wild type proteins, or, alternatively, mutant or variant proteins, including those with altered stability, activity, or other variant properties, or hybrid proteins to which foreign amino acid sequences, e.g., sequences that facilitate purification (e.g., a tag or fusion), have been added.

As used herein, the term “ligand” refers to an agent that binds a target protein. The agent may bind the target protein when the target protein is in its native conformation, when it is partially or totally unfolded or denatured, or when it is partially or totally aggregated. According to the present invention, a ligand is not limited to an agent that binds a recognized functional region of the target protein e.g. the active site of an enzyme, the antigen-combining site of an antibody, the hormone-binding site of a receptor, a cofactor-binding site, and the like. A ligand can also be an agent that binds any surface or internal sequences or conformational domains of the target protein. Therefore, the ligands of the present invention encompass agents that in and of themselves may have no apparent biological function, beyond their ability to bind to the target protein in the manner described above.

As used herein, the term “test ligand” refers to an agent, comprising a compound, molecule or complex, which is being tested for its ability to bind to a target protein. Test ligands can be virtually any agent, including without limitation metals, peptides, proteins, lipids, polysaccharides, nucleic acids, small organic molecules, and combinations thereof. Complex mixtures of substances such as natural product extracts, which may include more than one test ligand, can also be tested, and the component that binds the target protein can be purified from the mixture in a subsequent step.

The terms “multiplicity of molecules,” “multiplicity of compounds,” “multiplicity of samples”, or “multiplicity of containers” refer to at least two molecules, compounds, samples, or containers, respectively. The term “multiplicity” is used interchangeably herein with “plurality.”

The term “polarimetric measurement” relates to measurements of changes in the polarization properties of light and fluorescent emission. Circular dichroism and optical rotation are examples of polarization properties of light which can be measured polarimetrically. Measurements of circular dichroism and optical rotation are taken using a spectropolarimeter. “Nonpolarimetric” measurements are those that are not obtained using a spectropolarimeter.

The terms “spectral measurement” and “spectrophotometric measurement” refer to measurements of changes in the absorption of light. Turbidity measurements, measurements of visible light absorption, and measurement of ultraviolet light absorption are examples of spectral measurements.

“Stability” of a biological molecule refers to the ability of the biological molecule to resist aggregation and/or unfolding in conditions that tend to unfold or aggregate biological molecules. For example, a first protein is more stable than a second protein if the first protein is not significantly unfolded or aggregated at a temperature at which the second protein is significantly unfolded.

“Kinetics of unfolding” or “unfolding kinetics” or “denaturation kinetics” refers to the study of the extent of unfolding as a function of time. “Kinetics of aggregation” or “aggregation kinetics” refers to the study of the extent of aggregation as a function of time.

“Dynamics of unfolding” or “unfolding dynamics” or “denaturation dynamics” refers to the study of unfolding or denaturation as a function of environmental conditions in which a biological sample is disposed, including biochemical conditions. “Dynamics of aggregation” or “aggregation dynamics” refers to study of aggregation as a function of environmental conditions in which a biological sample is disposed, including biochemical conditions.

The terms “thermal change” and “physical change” encompass the release of energy in the form of light or heat, the absorption of energy in the form or light or heat, changes in turbidity, and/or changes in the polar properties of light. In exemplary embodiments, the terms include, for example, fluorescent emission, fluorescent energy transfer, absorption of ultraviolet or visible light, changes in the polarization properties of light, changes in the polarization properties of fluorescent emission, changes in turbidity, and changes in enzyme activity. Fluorescence emission can be intrinsic to a protein or can be due to a fluorescence reporter molecule (below). For a nucleic acid, fluorescence can be due to ethidium bromide, which is an intercalating agent. Alternatively, the nucleic acid can be labeled with a fluorophore (below).

Methods of the Invention

The invention is based at least in part on the observation that most proteins denature and/or aggregate when they are exposed to a variety of conditions, such as, for example, a change in temperature (increase or decrease) or non-physiological conditions. This unfolding is a consequence of the protein unfolding to an intermediate hydrophobic rate which may be followed by aggregation. Proteins may also aggregate directly from its folded state and may also remain in an unfolded state without proceeding to aggregation, depending on the environmental conditions. The invention provides methods and apparatus for studying the process of aggregation and also for studying the combined processes of unfolding and aggregation. Such studies can be summarized with quantitative measures, such as transition temperatures or rate constants, comparing the process of aggregation under different conditions, leading to, for example, identification of conditions which are favorable to stabilizing or crystallizing a particular biological molecule.

Most cellular proteins denature irreversibly, however some of the well studied proteins denature largely reversibly. There are two reasons for this, proteins such as ribonuclease, lysozyme, trypsin, etc., which can be isolated in large quantities and are normally used in most in vitro studies, are either secreted proteins, or intracellular proteins which are very stable and denature reversibly. Also, reversible unfolding can be analyzed by reversible thermodynamics, which is simpler to interpret. However, virtually all cellular proteins denature irreversibly at neutral pH and at the very high concentration present inside cells, which, indicates that understanding irreversible unfolding is important (Lepock, J. R., Frey, H. E. and Ritchie, K. P. (1993). Protein Unfolding in Intact Hepatocytes and Isolated Cellular Organelles During Heat Shock. J Cell Biol. 122, 1267-1276). This irreversibility is mainly a consequence of the irreversible aggregation that occurs after a reversible unfolding making the whole unfolding an irreversible process.

This three state, reversible-irreversible process can be modeled as:

$$nH {⇄}_{k - 1}^{{k}_{1}} nU {⟶}^{{k}_{2}} A \qquad 1$$

where H represents the native state, U the intermediate partially unfolded state, A represents the aggregated state, k₂ (or k_{agg}) is the rate constant of aggregation and n is representative of the degree of cooperativity during aggregation. Once the protein unfolds, hydrophobic residues are exposed, aggregation occurs and the protein becomes kinetically locked in the unfolded state. The unfolding process is described by k₁ (or kᵤ) and can be evaluated by bis ANS fluorescence or other methods like circular dichroism or differential scanning calorimetry. Light scattering is an example of a method that can be used to measure the process regulated by k₂. This three state model can often be simplified to a two state model of the form:

$$N {⟶}^{{k}_{app}} D \qquad 2$$

wherein N represents native state and D represents the denatured aggregated state and kₐₚₚ is an apparent rate constant. This approximation can be made if k₂ is of the same order of magnitude as k₁.

The calculated rate constants of unfolding kᵤ and aggregation k_{agg} are temperature dependent following the Arrhenius law:

k=Ae^{-Ea/RT} 3

and the logarithmic form is:

ln k=ln(A)−Ea/(RT) 4

where A is the Arrhenius pre-exponential factor, Ea the activation energy, R is the universal gas constant and T is the temperature in kelvin.

With the method and apparatus of the invention, rate constants (k) at different temperatures can be measured, which allows calculating the Ea of unfolding and aggregation respectively. When proteins are in the presence of molecules that interact with them, from the variations in the values of Ea's the interaction energy between the protein and the interacting molecule can be deduced.

In one embodiment, the invention provides methods and apparatus for identifying a condition that changes the stability of a biological molecule relative to its stability in a reference condition. Such methods involve characterizing aggregation and/or unfolding of a biological sample in one or more test conditions and/or one or more reference conditions and looking for changes in a property of aggregation and/or unfolding of the biological sample. Accordingly, the invention provides methods and apparatus for identifying conditions that increase the stability of a biological molecule and conditions that decrease the stability of a biological molecule.

The biological molecule can be, e.g., a peptide, polypeptide, protein (monomeric or multimeric); a nucleic acid, e.g., RNA, single, double, or triple stranded DNA, lipids, sugars, and combinations thereof. For example, the methods and apparatus of the invention permit the identification of conditions that stabilize a protein.

The condition can be, for example, a biochemical condition, pressure, electric current, time, concentration of the biological molecule, and presence of a test compound. A biochemical condition can be, for example, one relating to pH, ionic strength, salt concentration, oxidizing agent, reducing agent, detergent, glycerol, metal ions, salt, cofactor concentration, ligand concentration and coenzyme concentration. For example, the methods and apparatus of the invention permit the identification of salt concentrations that affect the unfolding and/or aggregation kinetics and dynamics of a protein.

In one embodiment, the biochemical condition is a solution comprising a compound, such as a compound of a small molecule library, and the methods and apparatus of the invention permit the identification of one or more compounds that bind to the biological molecule and thereby stabilize it. In an illustrative embodiment, the biological molecule is a protein and the method comprises identifying a ligand of the protein. Such a method can comprise incubating the protein with different solutions, each comprising a different potential ligand and/or concentration of potential ligand. The methods of the invention compare the relative affinity of the potential ligands to a given target, or relative affinity of a given ligand to a given protein in different test conditions.

In one embodiment, the method comprises (a) providing a composition comprising a biological molecule in a test condition; (b) bringing the temperature of the composition to an end temperature; and (c) determining the extent of aggregation and/or unfolding of the biological molecule as a function of time. In one embodiment, the end temperature of step (b) may be lower than the aggregation temperature of the biological molecule in a reference condition. The reference condition can be a condition in which the biological molecule is known to be relatively stable. The aggregation temperature of the biological molecule in the reference condition can be determined, e.g., by measuring the extent of aggregation as a function of increasing temperature, as known in the art and further described herein. The end temperature can be selected based on the desired rate of experimentation and the objectives of the study. The temperature can be about 1° C., 2° C., 5° C., 10° C., 15° C., 20° C., 25° C., or more degrees lower or higher than the transition aggregation temperature.

In an illustrative embodiment, a protein is provided in a solution A. Under these conditions, it is known, or was determined, that the aggregation temperature is about 50° C. The protein in mixed in a solution B, the solution with the protein is heated up to 45° C., and the extent of aggregation is measured.

In one embodiment, the compositions may be heated to their end temperature through a heat shock or by jumping the temperature, i.e., by bringing the temperature to the end temperature as fast as possible.

In certain embodiments, the extent of aggregation and/or unfolding is measured from a time point preceding the heat shock to the end temperature and continued after the time point at which the temperature of the composition attains the end temperature. In other embodiments, measurements of the extent of aggregation and/or unfolding are initiated about 1 second, 5 seconds, 10 seconds, 30 seconds, 45 seconds, 1 minute, 2 minutes, 3 minutes, 4 minutes, 5 minutes, 6 minutes, 7 minutes, 8 minutes, 9 minutes, or 10 minutes after the heat shock is initiated. In one embodiment, a plurality of essentially identical compositions are heat shocked in different tubes or wells of microwell plates and the measurements of extent of aggregation are initiated at different time points after the heat shock is initiated. This can be done, e.g., by heat shocking the tubes or microwell plates at different points, and starting the measurements of the different compositions essentially at the same time. Alternatively, the compositions are heat shocked at the same time, and the measurements of each composition are started after different intervals of time.

A heat shock can be made by different methods. For example, a tube at room temperature may be incubated in an environment that is at the desired end temperature under heat conducting conditions. Alternatively, a test solution at the desired end temperature is added to the protein that is in a minimum volume at a different temperature.

In another embodiment, the temperature is raised gradually, and the extent of aggregation is measured as a function of time. Measurements may be initiated at the time the temperature starts to rise, or at a time prior to, or after, the time the temperature starts to rise. The increase in temperature may be conducted, for example, at about one degree Celsius per minute.

In another embodiment, unfolding and aggregation are study in parallel as a function of time (kinetics) or temperature. To study kinetics, the compositions may be heated to their final temperature very rapidly. The extent of aggregation and unfolding are simultaneously measured as a function of time. Aggregation and unfolding curves as a function of time can then be generated. The aggregation kinetics of the said compositions can be characterized by one or more parameters such as rate of unfolding (kᵤ) and aggregation (k_{agg}). To study aggregation in parallel with unfolding as a function of temperature, thermal unfolding and aggregation curves can be generated by heating the compositions gradually, for example, one degree Celsius per minute, with measurements of extent of aggregation and/or unfolding initiated at the time the temperature starts to rise of at a time prior to or after the time the temperature starts to rise. The total process of unfolding and aggregation can be characterized by the transition temperatures of the two curves, respectively Tₘ and T_{agg}.

The extent of unfolding of a biological molecule can be determined according to a variety of techniques, such as calorimetry, circular dichroism, fluorescence emission (e.g., using intrinsic fluorescence or a fluorescence reporter molecule), fluorescence energy transfer, absorbance of ultraviolet or visible light, changes in the polarization properties of light, changes in the polarization properties of fluorescence emission, changes in turbidity, changes in enzyme activity, chaperone binding, or antibody binding (e.g., using different antibodies capable of recognizing the native or denatured state of the biological molecule). In an exemplary embodiment, fluorescence emission is used to determine the extent of reversible unfolding.

The fluorescence emission spectra of many fluorophores are sensitive to the polarity of their surrounding environment and therefore are effective probes of phase transitions for proteins (i.e., from the native to the unfolded phase). The most studied example of these environment dependent fluorophores is 8-anilinonaphthalene-1-sulfonate (1,8-ANS), for which it has been observed that the emission spectrum shifts to shorter wavelengths (blue shifts) as the solvent polarity decreases. These blue shifts are usually accompanied by an increase in the fluorescence quantum yield of the fluorophore. In the case of ANS, the quantum yield is 0.002 in water and increases to 0.4 when ANS is bound to serum albumin. ANS may be excited with a wavelength near 360 nm and produces a fluorescence emission that may be measured at 460 nm.

Fluorescence probe molecules are fluorophores that are capable of binding to an unfolded or denatured receptor and, after excitement by light of a defined wavelength, emitting fluorescent energy, such as UV light. Any fluorophore capable of binding to a denatured polypeptide may be used in accordance with the invention, including, for example, thioinosine, N-ethenoadenosine, formycin, dansyl derivatives, fluorescein derivatives, 6-propionyl-2-(dimethylamino)-napthalene (PRODAN), 2-anilinonaphtalene, and N-arylamino-naphthalene sulfonate derivatives such as 1-anilinonaphtalene-8-sulfonate (1,8-ANS), 2-anilinonaphthalene-6-sulfonate (2,6-ANS), 2-aminonaphthalene6-sulfonate, N,N-dimethyl-2-aminonaphthalene-6-sulfonate, N-phenyl-2-aminonaphthalene, N-cyclohexyl-2-aminonaphthalene-6-sulfonate, N-phenyl-2-aminonaphthalene-6-sulfonate, N-phenyl-N-methyl-2-aminonaph-thalene-6-sulfonate, N-(o-toluyl)-2-aminonaphthalene-6-sulfonate, N-(m-toluyl)-2-aminonaphthalene-6-sulfonate, N-(p-toluyl)-2-aminonaphthalene-6-sulfonate, 2-(p-toluidinyl)-naphthalene-6-sulfonic acid (2,6-TNS), 4-(dicyanovinyl) julolidine (DCVJ), 6-dodecanoyl-2-dimethylaminonaphthalene (LAURDAN), 6-hexadecanoyl-2-(((2-trimethylammonium-ethyl)methyl)amino) naphthalenechlo ride(PATMAN), nile red, N-phenyl-1-naphthylamine, 1,1-dicyano-2-[6-(dimethylamino) naphthalen-2-yl]propene (DDNP), 4,4′-dianilino-1,1′-binaphthyl-5,5′-disulfonic acid (bis-ANS), and DAPOXYL™ derivatives (Molecular Probes, Eugene, Oreg.).

Bis ANS is a fluorescent probe that does not bind to most native proteins but binds to hydrophobic surfaces of partially denatured proteins with a corresponding increase in fluorescence (Cardamone, M. and Puri, N. K. (1992). Spectrofluorometric assessment of the surface hydrophobicity of proteins. Biochem. J. 282, 589-593, Semisotnov, G. V., Rodionova, N. A., Razgulyaev, O. L., Uversky, V. N., Gripas, A. F. and Gilmanshin, R. I. Study of the molten globule intermediate state in protein folding by a hydrophobic fluorescent probe. (Biopolymers, 31, 119-128).

When using a fluorophore, the fluorophore is added to the composition comprising the biological molecule prior to initiating the denaturing process, e.g., prior to a change in temperature or addition of a chemical denaturant. For example, bis-ANS can be added to a composition comprising a biological molecule, the composition is mixed, the composition is subjected to a heat shock to the end temperature, and the fluorescence of the composition is measured over time.

Any of the variety of fluorescence emission imaging systems known to those skilled in the art may be used to monitor the extent of reversible unfolding of a biological molecule. For example, CytoFluor II fluorescence microplate reader (PerSeptive Biosystems, Framingham, Mass.) is an example of a fluorescence imager that may be used in accordance with the invention. A Charge Coupled Device Camera (“CCD camera”) may also be used to measure fluorescence emission.

Intrinsic tryptophan (Trp) fluorescence is an alternative method for determining the extent of unfolding of a polypeptide. The intrinsic Trp residues of a polypeptide may be excited with light near 280 nm resulting in a fluorescence emission near 350 nm. Such Trp fluorescence excitation may be achieved using a Xenon-Arc lamp, such as the Biolumin 960 (Molecular Dynamics).

If the biological molecule is a nucleic acid, the extent of unfolding may be determined using light spectrophotometry. Unfolding is measured by determining the change in hyperchromicity, which is the increase in absorption of light by polynucleotide solutions due to a loss of ordered structure, for example, in response to an increase in temperature. Fluorescence emission may also be used to measure the extent of unfolding of a polynucleotide. The nucleic acid may be labeled with ethidium bromide or a fluorophore and fluorescence spectrometry may be used to monitor the level of fluorescence emission. Fluorescence resonance energy transfer may also be used in accordance with the invention. In this approach, the transfer of fluorescent energy, from a donor fluorophore on one strand of an oligonucleotide, to an acceptor fluorophore on the other strand, is measured by determining the emission of the acceptor fluorophore. Unfolding diminishes or prevents the transfer of fluorescent energy.

The extent of aggregation of a biological molecule can be measured spectrophotometrically, e.g., through measurements of light scattering or turbidity, or by electron microscopy, velocity sedimentation, centrifugation, or filtration. In an exemplary embodiment, the extent of aggregation is measured by determining the optical density (“OD”) of a sample using ultraviolet or visible light. A higher optical density denotes larger particles and thus a greater extent of aggregation of the biological molecule in the sample.

In an exemplary embodiment, the formation of aggregates is followed by static light scattering. This is possible because the intensity of the light scattered is proportional to the size of the particles in suspension. The size of these particles increased from a few nm, which is the normal size of a protein in solution to sizes on the order of μm when the proteins have aggregated.

In exemplary embodiments, the light source for light scattering may be one or more of the following: a laser (e.g., a monochromatic, intense, well defined beam of light), a light emitting diode (LED), a cluster of LEDs, a white light source, a monochromatic light source, an incandescent light source, a Xenon-arc lamp, a tungsten-halogen lamp, an ultraviolet light source, a luminescent light source, and/or a low intensity light source with an intensity in a range of 1.5 to 2.0 μW/mm².

Depending on the wavelength of the incident light source and the dimensions of the particle, this scattered light can show very characteristic intensity patterns. Small molecules scatter light equally in all directions. On the other hand, particles at least as big as the wavelength of the incident light scatter more in certain directions than others.

The Raleigh ratio R(θ)=MP(θ)K*c describes the absolute intensity scattered at an angle θ in excess of the light scattered by the pure solvent. M is the molecular mass of the scattering particle and proportional to its size, c is the concentration and P(θ) is the form factor (ratio of scattered intensity at angle θ to intensity at angle 0), K is an optical constant and contains the refractive index of the solvent, Avogadro's number, the wavelength of the incident light, and the specific refractive index increment of the sample molecules. For simple mathematical reasons the maximum intensity of the light scattered can be measured at a 90° angle.

Additional information regarding spectrophotometry and spectrofluorometry can be found, e.g., in Bashford, C. L. et al., Spectrophotometry and Spectrofluorometry: A Practical Approach, pp. 91-114, IRL Press Ltd. (1987); Bell, J. E., Spectroscopy in Biochemistry, Vol. I, pp. 155-194, CRC Press (1981); Brand, L. et al., Ann. Rev. Biochem. 41:843 (1972); Ozaki, H. et al., Nucleic Acids Res. 20:5205-5214 (1992); Clegg, R I M. et al., Proc. Natl. Acad. Sci. U.S.A. 90:2994-2998 (1993); Clegg, R. M. et al., Biochemistry 31:4846-4856 (1993); Lee, M. et al., J. Med. Chem. 36:863-870 (1993); U.S. Pat. Nos. 6,303,322; 5,858,277; 6,270,954; 5,854,204.

A person of skill in the art will recognize that where appropriate, unfolding and aggregation can also be measured by other methods, such as non-spectroscopic methods. For example, methods for detecting unfolding of biological molecules include methods which detect the presence of folded and/or unfolded biological molecules by virtue of binding of another molecule to the folded or unfolded biological molecules. Exemplary techniques include the use of antibodies which specifically recognize epitopes that are exposed only in a protein when it is unfolded or alternatively, which are exposed only in a protein when it is folded. Such techniques are further described, e.g., in U.S. Pat. No. 5,679,582.

The measurements of extent of aggregation and/or unfolding can be conducted about every 10 seconds, 20 seconds, 30 seconds, 40 seconds, 50 seconds, 1 minute, 2 minutes, 3 minutes, 4 minutes, 5 minutes, or more. As little as one or two measurements may be sufficient in certain embodiments. In other embodiments, 5, 10, 15, 20, 30, 50 or more measurements are conducted over time on one or more samples. Measurements may be conducted until the maximum aggregation is attained, e.g., when the fluorescence or light scattering has attained a maximum and is not further increased with time.

Measurements can be conducted in an automated fashion. For example, one or a plurality compositions can be incubated in wells of a microwell plate; the plate is heated; the plate is then illuminated in an automated fashion at particular time intervals; and fluorescence emission or light scattering is measured in an automated fashion over time. Measurements can be taken from the top of the plate.

The results of the measurements can then be collected, e.g., in an automated fashion. The results can be transmitted to a computer readable medium or a computer. Analysis of the results can be conducted on a computer. The computer may further comprise results obtained from other assays and may contain reference data.

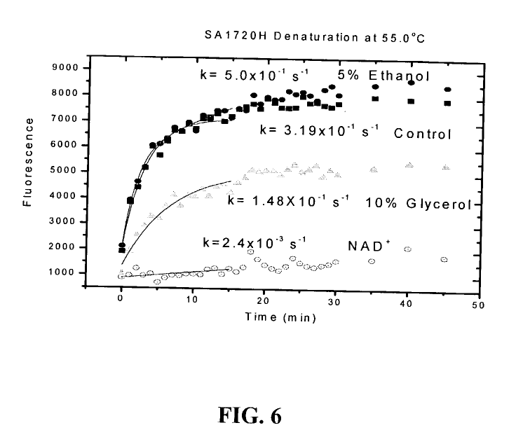

In embodiments in which fluorescence or light scattering is measured, the results can be plotted as fluorescence or light scattering as a function of time (“time scale method”; see, e.g., FIG. 6). The curve, or one or more points thereof, may then be compared to that of the biological molecule in different conditions, e.g., in a different salt concentration. In one embodiment, the curve, or one or more points thereof, is compared with that of the biological compound in a reference condition.

In other embodiments, the rate constant of unfolding (kᵤ) or aggregation (k_{agg}) are determined from the results obtained. kᵤ and k_{agg} can be obtained from the fitted exponential growth portion of the curves. A lower kᵤ or k_{agg} of a biological molecule in a first condition relative to that of the biological molecule in a second condition indicates that the biological molecule is more stable in the first condition relative to the second condition.

In another embodiment, the energy of activation (Ea) can be deduced based on the Arrhenius law (see above). For example, the interaction energy between a protein and an interacting molecule can be deduced by determining the change in the value of Ea's when the protein is in the presence of the interacting molecule. Other variables that can be deduced include the maximum scattered intensity (Iₘₐₓ). Iₘₐₓ can be used to evaluate conditions that induce or prevent aggregation with or without inducing destabilization.

The amount of native protein remaining after exposure to some temperature (T) for a time (t) is:

N(t)=N₀e−^{kapp(T)t} 5

where N₀ is the amount of native protein at time t=0.

Making N(t)=No/2 and obtaining kₐₚₚ from the experimental fit, the time required to obtain 50% aggregation at the temperature T can be calculated as a measure of stability.

A computer with appropriate algorithm can derive some or all of these variables for each biological molecule at each temperature in each condition. A comparison readily indicates which conditions provide the most stabilizing effect on the biological molecule.

In an exemplary embodiment, both the extent of unfolding and aggregation are determined. Measuring both factors may be relevant to complete understanding of aggregation kinetics and dynamics, particularly in cases where macromolecules unfold prior to proceeding to aggregation. Measuring both parameters is preferably conducted essentially simultaneously. For example, a tube or microwell plate may be irradiated alternatively with a UV light to detect the extent of unfolding and with another light source to detect light scattering. In this embodiment, the fluorescence emission and light scattered are also measured essentially simultaneously, e.g., with a CCD camera. Thus, in such an embodiment, the extent of unfolding and the extent of aggregation are essentially determined simultaneously.

In another embodiment, two identical tubes, or microwell plates, or portions of microwell plates, are used. One tube, or microwell plate, or portion thereof, is illuminated with UV light for detecting the extent of unfolding, and the other tube, or microwell plate, or portion thereof, is illuminated with a light source for detecting the extent of aggregation. Results can be measured and processed simultaneously.

After having obtained the measurement of extent of aggregation and/or unfolding at one temperature, the temperature can be increased, e.g., jumped or gradually increased, to another temperature, e.g., a higher temperature. For example, once maximum levels of aggregation and/or unfolding are obtained at a particular temperature, the temperature may be increased by 1° C., 2° C., 3° C., 5° C., 7° C., 10° C., 15° C., 20° C., 25° C., or more degrees. Measurements can be continued when the temperature is jumped or measurements can be interrupted during the jump in temperature, and reiterated a certain time after the beginning of the temperature jump.

In one embodiment, the extent of aggregation and/or unfolding are determined at several temperatures essentially simultaneously. For example two essentially identical compositions comprising a biological molecule can be exposed to different temperatures. The essentially identical compositions can be in different tubes or microwell plates. In one embodiment, the compositions are in different wells of a microwell plate. For example, one or more individual wells (including e.g., a row of wells or entire plate) can be exposed to one temperature and another one or more wells can be exposed to another temperature. Measurements of the extent of aggregation and/or unfolding in the tubes or wells exposed to the different temperatures can be conducted simultaneously over time.

In another embodiment, a gradient of temperature may be created within an individual tube or plate. In an illustrative embodiment, one end of a plate containing a composition comprising a biological molecule is at a first temperature and the other end of the plate is at a second temperature. A gradient of temperature may be formed between these two temperatures. Measurements can be taken at both ends of the plate as well as at locations between the two ends for measuring effects at an intermediate temperature.

The methods and apparatus of the invention are easily adaptable to high throughput screenings. In an illustrative embodiment, different conditions are tested simultaneously for one biological molecule, e.g., in a multiwell plate. All the conditions can be tested at the same end temperature. All the conditions can also be tested at a plurality of different end temperatures. For example one 384 well plate contains the same biological molecule in 384 different conditions, i.e., one condition per well, and is incubated at a first temperature. A second, essentially identical plate can be incubated at a different temperature. The invention also provides methods for simultaneously evaluating different biological molecules in one or more conditions. For example, an assay can comprise evaluating a first protein and proteins which differ from the first protein in one or more amino acid differences. In an illustrative embodiment, a microwell plate has columns of different biological molecules and rows of different conditions.

Multiwell plates that can be used exist in numerous formats, e.g., 24 well plates (4×6 array), 96 well plates (8×12 arrays), 384 well plates (16×24 array), 864 well plates (24×36 array), and 1536 well plates (32×48 array). Accordingly, the invention provides methods for simultaneously evaluating the stability (by the extent of unfolding and/or aggregation) of at least about 5, 10, 25, 50, 100, 250, 500, 1000, 2500, 5000, 10,000 or more conditions and/or biological molecules.

In a particular embodiment, the invention provides methods for identifying ligands of biological molecules, such as proteins. A method may comprise providing a composition comprising a target protein and a test ligand. In an exemplary embodiment, the method comprises heat shocking the temperature of the composition, optionally, to an end temperature that is lower than the aggregation temperature of the protein without the test ligand. The method may further comprise subjecting the composition to incident illumination which will result in scatted light proportional to accumulation of aggregates prior to, at the same time, and/or after beginning the heat shock. Scattered light is detected over time until it reaches a maximum. The scattered light intensities can then be plotted as a function of time, and the curve can be compared to a curve of the protein that was not incubated with the test ligand. The rate of aggregation (k_{agg}) can also be derived from the curve, and compared to the k_{agg} of the protein in the absence of the test ligand. A lower k_{agg} in the presence of the test ligand indicates that the ligand binds to the protein. Using the same method, a library of potential ligands can readily be tested. One or more test ligands can be incubated with a target protein and the measurements of scattered light can be conducted simultaneously.

In other embodiments, the extent of aggregation and/or unfolding may be measured as a function of varying temperature instead of, or in addition to, a function of time. For example, the sample may be illuminated with a light source and the light scattering is measured (e.g., with a CCD camera) as the temperature of the compositions are varied (either increased or decreased) at a controlled rate. In various embodiments, the temperature may be increased or decreased in a continuous or stepwise fashion. Additionally, when using a multiplicity of samples, the temperature of essentially all samples may be controlled in bulk or the temperature of one or more samples may be controlled separately from other samples. In one embodiment, the light source may be a laser. In yet other embodiments, both the extent of unfolding and the extent of aggregation are determined to identify ligands binding to a target protein.

In other embodiments, the methods can be used to identify compounds that modulate the interaction between a biological molecule and a ligand. For example, the method may comprise providing a composition comprising a protein, a ligand, and a test compound. The composition is heat shocked, optionally, to a temperature that is lower than the aggregation temperature of the protein-ligand complex, and the fluorescence emission or light scattering is measured as a function of time. The kᵤ or k_{agg} of the composition with the test compound can be determined. A lower kᵤ or k_{agg} in the presence of the test compound relative to the absence of the test compound indicates that the test compound inhibits the interaction between the protein and the ligand.

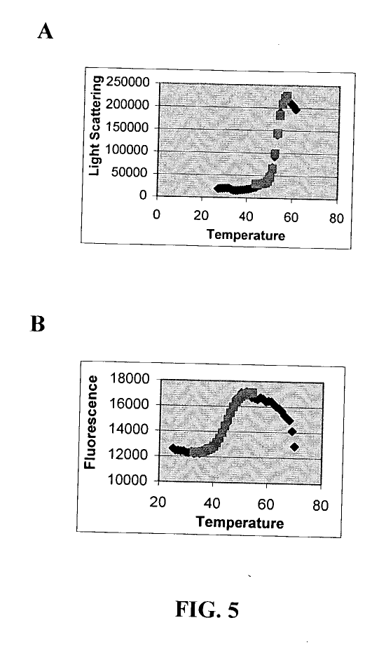

The invention also provides methods for identifying a condition in which a biological molecule has a different stability relative to its stability in a reference condition comprising (a) providing a composition comprising a biological molecule in a test condition; (b) increasing the temperature of the composition over time; and (c) determining the extent of unfolding and aggregation of the biological molecule in an essentially simultaneous manner during the increase in temperature. In one embodiment, identical compositions are present in different tubes or microwell plates and one tube or microwell plate is monitored for the extent of unfolding, whereas the other tube or microwell plate is monitored for the extent of aggregation. In an exemplary embodiment, the extent of unfolding and aggregation are determined on the same sample, in an alternative fashion. For example, a sample is alternatively illuminated with a UV light (for determining the extent of unfolding) and a light source for light scattering (for determining the extent of aggregation) and the fluorescence emission and light scattering are detected alternatively with different detectors, or simultaneously with the same detector, e.g., a CCD camera. The results can then be analyzed. In one embodiment, the results are plotted as the amount of fluorescence and/or light scattered as a function of temperature (“temperature scale method;” see, e.g., FIG. 5). The curves or at least some points thereof can be compared to the curve or points thereof of the biological molecule in a reference condition, to determine whether the test condition stabilized or destabilized the biological molecule relative to the reference condition. In another embodiment, the Tₘ (melting temperature) and T_{agg} (“aggregation temperature”) are derived from the sigmoid portion of the curves. A higher Tₘ and T_{agg} of a biological molecule in a test condition relative to the Tₘ and T_{agg}, relatively, of the biological molecule in a reference condition indicates that the test condition stabilizes the biological molecule. The maximum fluorescence (I_{f}) and scattered intensity (I_{agg}) can also be obtained for each protein. I_{agg} is an indication of the magnitude of the aggregation. Iₘₐₓ is a measure to evaluate conditions that may induce or reduce aggregation with or without protein destabilization.

This particular embodiment can also be used to screen a library of compounds to identify one or more compounds that bind to a target biological molecule. Similarly, the method can be adapted to screen for compound which inhibits the interaction between a biological molecule and a ligand.

Thus, the invention provides useful methods and apparatus for at least the following: (i) to conduct biophysical characterization of protein by generating aggregation and/or unfolding curves as a function of time and/or temperature; (ii) to conduct biophysical characterization of a protein by generating both unfolding and aggregation curves as a function of time and/or temperature; (iii) to characterize protein dynamics by defining precisely numerical measures such as the aggregation temperature (T_{agg}) of aggregation, rate of aggregation (k_{agg}), melting temperature (Tₘ) and rate of unfolding (kᵤ) as characteristic biophysical properties of individual proteins; (iv) to identify substances or conditions that affect stability of any individual protein by shifting, by virtue of their presence, biophysical properties such as Tₘ, T_{agg}, kᵤ and k_{agg}; (v) to identify substances or conditions that without affecting the stability of any individual protein can increase the size or number of protein aggregates; (vi) to identify substances or conditions that without affecting the stability of any individual protein can decrease the size or number of protein aggregates; (vii) to identify substances that prevent protein aggregation and precipitation; (viii) to identify substances or conditions that stimulate protein aggregation and precipitation; (ix) to measure rates of protein unfolding kᵤ at different temperatures; (x) to measure rates of protein aggregation k_{agg} at different temperatures; (xi) to measure the activation energy of protein unfolding Ea_{d}; (xii) to measure the activation energy of protein aggregation Eagg_{d}; (xiii) to measure the interaction energy between a protein and a molecule that interacts with it.

A person of skill in the art will recognize that denaturing conditions other than heat can be used according to the methods of the invention for identifying conditions that stabilize or destabilize a biological molecule. For example, a composition comprising a biological molecule in a test condition can be subjected to a denaturing agent, such as a chaotropic agent (e.g., urea and guanidium), and the extent of aggregation and/or unfolding determined as a function of time or temperature. In another embodiment, mechanical denaturation, such as, for example, sonication may be used in accordance with the methods and apparatus disclosed herein. Such assays will provide information on conditions which stabilize a biological molecule with respect to denaturing conditions other than heat.

Apparatus of the Invention

When light encounters particles in its path, the electric field of the electromagnetic radiation displaces the particles, causing them to oscillate around their equilibrium positions. The oscillating particles act as secondary sources, re-radiating or scattering the incident energy. In elastic scattering, the scattered light is at the same frequency as the incident radiation. This phenomenon is the most dominant form of scattering and includes Rayleigh and Mie scattering. Inelastic scattering is the phenomenon of the molecules emitting radiation at their own characteristic rotational and vibrational frequencies. This includes the phenomenon of Raman scattering.

The scattering from molecules and very tiny particles (<1/10 wavelength) is predominantly Rayleigh scattering, which accounts for the blue color for a clear sky (i.e. the blue end of the electromagnetic spectrum is of short wavelength). For particle sizes larger than a wavelength, Mie scattering becomes dominant. This phenomenon tends not to be frequency dependent resulting typically in white scattered light. Examples of Mie scattering include the white glare around the sun when a lot of particulate is present in the air, as well as mist and fog.

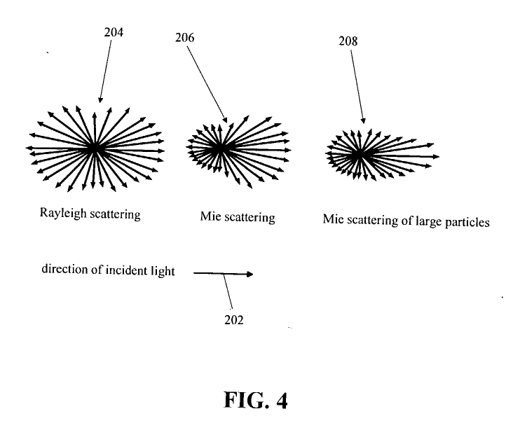

FIG. 4 shows typical patterns of Rayleigh and Mie scattering. The arrows in each pattern represent light scattered from a particle located at the origin of the arrows, with the length of an arrow representing the relative intensity of light scattered in the direction of the arrow. The incident direction of the light for each pattern is indicated by arrow 202. Rayleigh scattering, as indicated by arrows 204, tends to be weakest in the direction perpendicular to the incident radiation but is roughly uniform elsewhere. Mie scattering, as indicated by arrows 206, tends to be strongest in the same direction as the incident light. As particles become larger, Mie scattering can result in a narrower dispersion in the direction of the incident light, as indicated by arrows 208. This narrowing may pose problems in measuring the scattered light.

If particle size (r) is much smaller than the incident light wavelength (λ) the system emits radiation as an electric dipole. As known in the art, the Rayleigh ratio is defined as the ratio of the scattered light intensity (I) to the incident light intensity (I₀) measured at a given angle (θ) and distance (r) from the scattering volume:

I/I₀=R_{θ}(1+cos² θ)/r² 6

Scattering intensity is dependent upon a number of particle and solvent parameters including particle polarizability (α), permittivity (ε), density (ρ) and molecular weight (A):

R_{θ}=½(ρN_{A}/M)(π²α²/ε²λ⁴)), 7

where N_{A} is Avogadro's number. The strong dependence on λ indicates much more intensive scattering at short wavelength, e.g., ultraviolet (UV) and visible light in contrast to near-infrared (IR) and IR.

For practical measurements the following form of the Rayleigh equation is used:

$$\frac{KC}{{R}_{θ}} = ( \frac{1}{M} + 2 {A}_{2} C ) [ 1 + \frac{16 {π}^{2} {R}_{g}^{2}}{3 {λ}^{2}} {sin}^{2} ( \frac{θ}{2} ) ] , \qquad 8$$

where C is the particle concentration in solution, A₂ is the 2^{nd} virial coefficient, indicative of solute-solvent interaction, R_{g} is the radius of gyration of the particle; K is an optical parameter equal to 4π²n²(dn/dC)²/λ⁴N_{A}, where n is the solvent refractive index and (dn/dC) is the analyte specific refractive index increment. The angular dependent portion of the second term in Equation 8 arises from interference effects due to multiple scattering from a single particle. For particles much smaller than the wavelength of the incident radiation, this term goes to zero and the angular dependence of the scattered light vanishes. Under these conditions, the absolute molecular weight is determined from the concentration dependence of the Rayleigh ratio and angular dependent data need not be obtained.

For larger particles, it is still the concentration dependence that leads to molecular weight, but the interference effects, as characterized by the second term of Equation 8, must be accounted for. At this point multi-angle instruments may become necessary. As a rule of thumb, the size cutoff for angle independent Rayleigh scattering is R_{g}<λ/20. Typical wavelengths used in standard static light scattering (SLS) equipment include wavelengths in the range of 600-800 nm, which provides a 30-40 nm upper limit for single-angle particle molecular weight determination. However, when considering protein studies, the native, folded forms of proteins can be within the above limit, while aggregations of particles may typically be larger. For particles sizes r greater than λ/20, incident radiation can induce multipole moments and the classical dipole approximation described above becomes inappropriate. Alternatively, a technique of dynamic light scattering (DLS) may be used to obtain the “size” of the particle.

As is known in the art, in DLS one measures the time dependence of the light scattered from a very small region of solution. Typical time scales can range from tenths of microseconds to milliseconds. Fluctuations in the intensity of the scattered light are related to the rate of diffusion of molecules in and out of the region being studied (Brownian motion). The measured light signals contain contributions from the slower movement of large particles as well as from the faster fluctuations of small size particles.

Signals can be analyzed in terms of an autocorrelation function or a fast Fourier transform (FFT) and plotted as maps of intensity vs. time, or intensity vs. frequency, respectively. Each mono-dispersed population (particles of a single size) produces its own unique correlation function—a single exponential decay:

C(τ)=Ae^{-2Γt}+B, 9

where A and B are instrumental constants. In turn, Γ=q²D, where q is a scattering vector,

q=(4πn/λ)Sin(θ/2), 10

and D is a translational diffusion coefficient related to a hydrodynamic radius, Rₕ of a spherical particle and solvent viscosity, η by Stokes-Einstein equation:

D=k_{b}T/(3πηT). 11

Mixtures of more than one size population produce the sums of exponentials. The hydrodynamic radius, Rₕ characterizes individual mono-dispersed fractions in the solvent. In general, proteins are not spherical, and their apparent hydrodynamic size depends on their shape (conformation) and mass. Therefore, the apparent hydrodynamic size can differ significantly from the true physical size, and may not be a reliable measure of molecular mass.

DLS data is commonly presented as the fraction of particles versus their diameters. Sets of mono-dispersed standards (polystyrene particles of known size) are typically used to calibrate the scale of the sizes. The strength of DLS lies in its ability to analyze samples containing broad distributions of species of widely differing molecular masses, (e.g. a native protein and various sizes of aggregates), and to detect very small amounts of the higher mass species (<0.01% in many cases).

Light scattering from macroscopic particles can be treated in terms of Mie scattering theory. A scattering particle is idealized as a sphere with a particular geometrical size. This sphere redirects incident photons into new directions and so prevents the forward on-axis transmission of photons, thereby casting a shadow. The size of the scattering shadow is called the effective cross section (σ, cm²) and can be smaller or larger than the geometrical size of the scattering particle (πr², cm²). The effective cross section is related to the geometrical size by a proportionality constant referred to as the scattering efficiency Q:, i.e., σ=Q(πr²). The scattering coefficient μ(cm⁻³) describes a medium containing many scattering particles at a concentration described as a volume density σ(cm⁻³). The scattering coefficient is the cross-sectional area per unit volume of medium: μ=ρσ.

Even though at the molecular level, as described above, incident light photons are scattered away from the direction of incidence. The resulting Mie scattering pattern favors the incident direction, as shown in FIG. 4. This has to do with constructive and destructive interference of scattered light. It is well known in optics that interference is totally constructive only in the forward direction. Mie theory provides a mathematically rigorous algorithm to describe this anisotropic angular distribution of light scattered by macromolecules and to calculate angular distribution as a function of wavelength and refractive index of the spheres. Typically, scattered light has a few maxima in angular distribution with strong preference of scattering into small angles relative to incident light, e.g., <45° from incidence, and preferably in a range between 15° to 30°, as shown in FIG. 4. The scattering is stronger if incident light is polarized in the plane formed by the light source, the scattering object and the detector. In practical applications, Mie scattering theory is used to determine particle size distributions in the range of 50 nm to 2 mm, diagnostics and imaging of tissues, e.g. density of lipid membranes in the cell, size of nuclei, presence of collagen fibers and status of hydration in the tissue.

Whichever phenomenon of light scattering is being measured, the light source may be critical for obtaining adequate measurements. For Rayleigh and dynamic light scattering, high intensity and coherent incident illumination can provide greater sensitivity. As a result, a laser light may be chosen as the source of illumination in one embodiment. However, the use of individual lasers to illuminate individual samples can quickly become too costly as the number of samples increases. The complexity involved in designing such an instrument can also be daunting in optics and electronics, resulting in a solution with great limitations in scalability. For example, the size of individual laser emitters can limit how closely they can packed, thus limiting the sample density. In one embodiment, non-coherent incident illumination with intensities in a range as low as 1.5-2.0 μW/mm² can provide sufficient illumination to detect Mie scattering and effectively monitor the aggregation of proteins. At such low levels of intensity, inexpensive non-coherent light sources, e.g., light emitting diodes (LED's) and filament-based light bulbs, can provide the necessary illumination.

In addition to the above considerations for the light source, the elimination of crosstalk can be an important consideration. Crosstalk can occur when reflected light from one sample can illuminate one or more adjacent samples. To inhibit crosstalk when measuring scattered light from a multiplicity of samples arranged in rows and columns next to each other, adjacent samples can be contained in wells that provide optical isolation between the wells. In one embodiment, microtiter plates containing arrays of wells can be used. The walls between the wells of the microtiter plates can be fabricated of an opaque plastic, e.g., black, to optically isolate the wells from one another. The bottom of the wells can be clear, or otherwise allow for the passage of light, so as to allow illumination from the light source to enter the wells through the bottom of the wells.

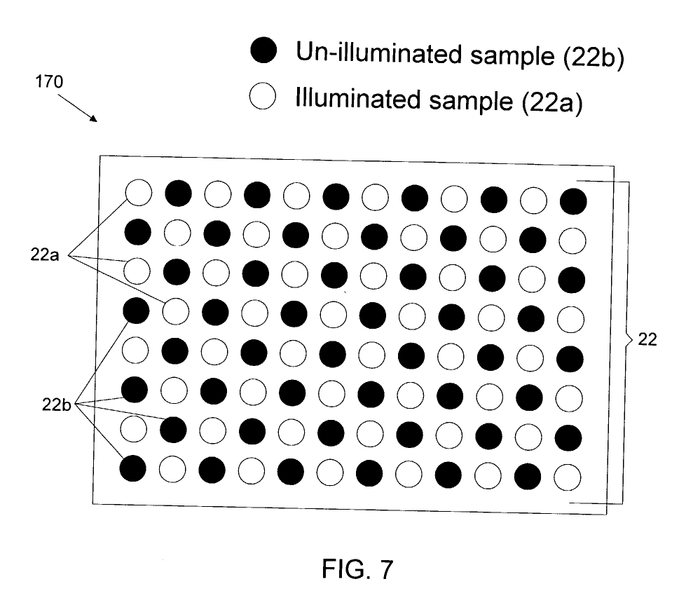

In one embodiment, selective illumination of the samples can be used to inhibit crosstalk. For example, in an array of sample wells, a single well can be illuminated, or wells in a chosen pattern can be simultaneously illuminated. The illumination pattern can be chosen so as to ensure that the light scattered from one illuminated sample does not add to the illumination of other illuminated samples. FIG. 7 illustrates an exemplary illumination pattern 170 for an array of sample wells 22, wherein illuminated sample wells 22a are alternated with un-illuminated samples wells 22b, only a portion of which are identified for clarity. In effect, the spacing of the illuminated wells can optically isolate the illuminated wells. Once measurements of the scattered light from the first illuminated well or pattern of wells are taken, another well or pattern of wells can be illuminated. By using selective illumination, the costs associated with providing sample wells fabricated with opaque or coated walls can be avoided.

The light scattered from the illuminated samples can be measured by various means known to those skilled in the art, including machine vision, photomultipliers and CCD's. In one embodiment, a machine vision system with the appropriate optical magnification can be used such that every sample in the experiment can be captured. The image can be captured shortly after the samples (or a subset of which) are illuminated. The image typically can be stored for analysis after the experiment. The first stage of analysis typically can involve image processing to determine the intensity of the light scattered by each sample.

As previously described, uniform and predictable heating of the samples may be required to obtain meaningful results. Preferably, the samples in the wells, including the control and test samples, can be heated to within 0.5° C. of each other, or can be heated in a selected pattern with a known temperature distribution. Heating of the samples can be provided by a water bath, Peltier heating, and/or other means as may be known in the art.

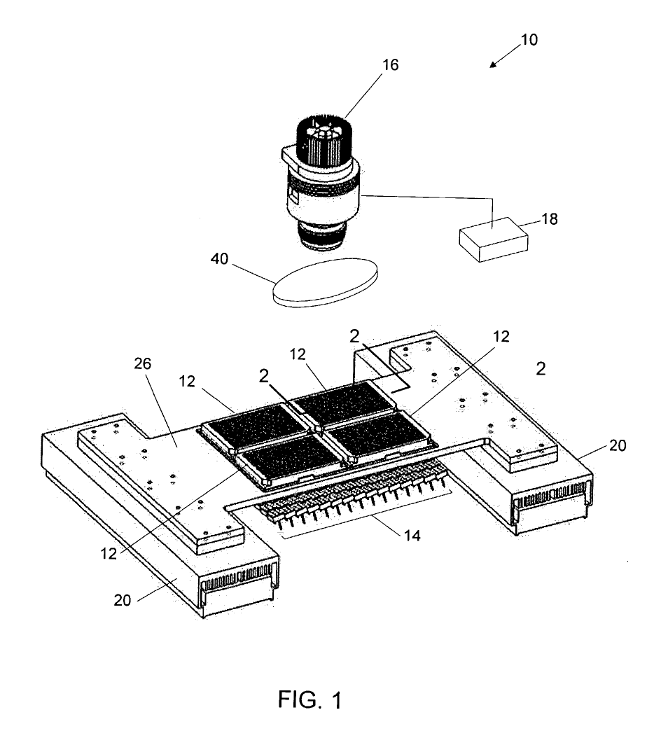

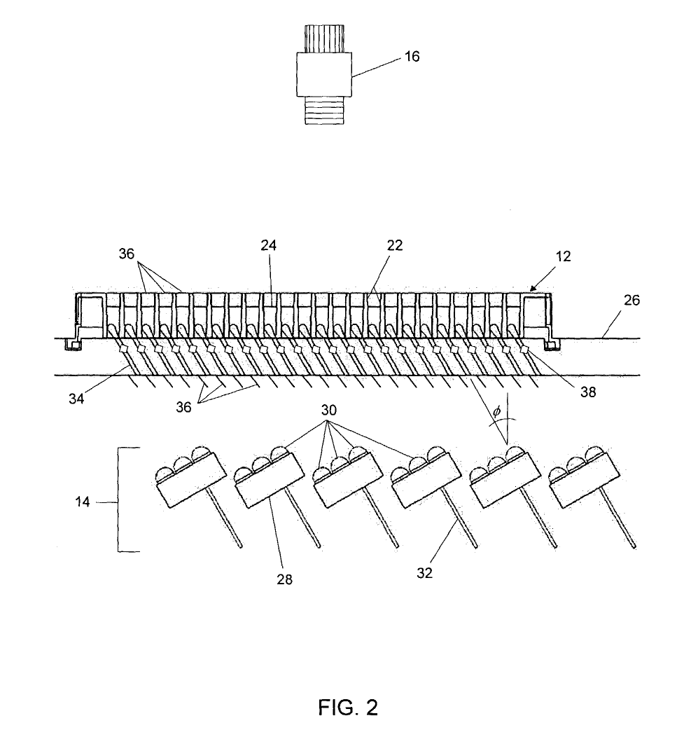

Referring now to FIG. 1, an isometric view schematically illustrating selected components of an exemplary apparatus 10 is shown. It can be understood by those of skill in the art that different configurations of apparatus 10 can be contemplated, which configuration may not be limited by the description and illustrative figures of apparatus 10 provided herein. Apparatus 10 can be configured to support one or more composition samples. In the exemplary embodiment of FIG. 1, the molecular samples can be contained in microtiter trays 12 that may each have an array of sample wells for the molecular samples, as further described in relation to FIG. 2.

A light source 14, e.g., a laser, an incandescent light source, a Xenon-arc lamp, or a tungsten-halogen lamp, can illuminate the samples. For the exemplary embodiment illustrated in FIG. 1, the light source can include an array, or cluster, of light emitting diodes (LED's) that can selectively illuminate the sample wells in trays 12. The illuminated samples can scatter the incoming light such that detector 16 can measure, or otherwise obtain a signal corresponding to the intensity of the light from source 14 scattered in the direction of detector 16 by the illuminated samples. A processor 18 can receive the signals from detector 16 and can determine the intensity of scattered light from the illuminated samples. Based on equations 6-11, processor 18 can determine a measure of the aggregation in the illuminated samples, as previously described.

Apparatus 10 can include one or more heaters 20 that may be used to uniformly, or selectively heat the sample wells of trays 12. In one embodiment, the heaters 20 can provide a temperature gradient across the trays 12. In another embodiment, trays 12 may be contained in a water bath, which can be heated by heaters 20. As previously described, heaters 20 can include other heating means as may be known in the art, e.g., Peltier heaters.

Referring now to FIG. 2, there is schematically illustrated a partial cross-sectional view of apparatus 10, taken at line 2-2 of FIG. 1. For the exemplary embodiment of FIG. 2, tray 12 can include an array of sample wells, one of which is labeled 22 in FIG. 2. The wells can contain molecular samples, as may be indicated by 24 in FIG. 2. It can be understood that, in lieu of tray 12, apparatus 10 can include individual molecular samples that can be supported on frame 26.

For the exemplary embodiment of FIG. 2, light source 14 includes an array of LED clusters 28, with each cluster having a grouping of LED's 30. Power for the LED's 30 can be provided via connectors 32, such that LED's 30 and/or clusters 28 can be individually and selectively powered. It can be understood that light source 14 can be an array of individual LED's 30, though clustering of LED's 30 may provide cost savings.

As previously noted, light scattering measurements are preferably obtained with light at an incident angle of less than 45° and preferably in a range of 15°-30°. LED's 30 can be oriented to provide light into sample wells 22 at an incident angle φ, with φ<45° and preferably in a range of 15°-30°, in order to achieve the dual purpose of (i) taking measurements in the range of angle providing maximum intensity of scattered light and (ii) avoiding detection of incident illumination. In order to control the beam geometry and dimensions of illumination on every sample and in order for the incident angle to remain consistent for each of the sample wells 22, a light guide 34 can be provided for each sample well 22. The light guides 34 may include parallel bores or passages through frame 26 which can be aligned with the chosen incident angle φ. In one embodiment, light guides 34 can include optical fibers. Light guide 34 can also contribute to elimination of crosstalk and can be configured to project discrete, uniform “spots” of light on each sample at a roughly constant location with respect to each sample.

The number of LED's 30 may correspond with the number of sample wells, so that each LED 30 can illuminate one sample well 22. However, cost and/or size limitations may preclude having one light source for each sample well. Furthermore, obviation of a direct relationship between the light source and sample density makes the solution more scalable in the sense that sample density may be varied without changing the light source. In the embodiment of FIG. 2, it can be seen that one LED 30, or a cluster of LED's 28 may illuminate more than one sample well 22. When it is desired to illuminate selected sample wells 22, occluding means can be provided to selectively block light from source 14 from entering other sample wells. Such occluding means can include one or more devices, such as operable shutters 36 and/or optical switches 38, as shown in FIG. 2, or other devices as may be known in the art, e.g., polarizing filters, or liquid crystal arrays. It can be understood that such occluding means can be additionally and/or alternatively provided to selectively block light from other than the selected sample cells from reaching the detector 16. For example, shutters 36 may be positioned between the sample wells 22 and detector 16.

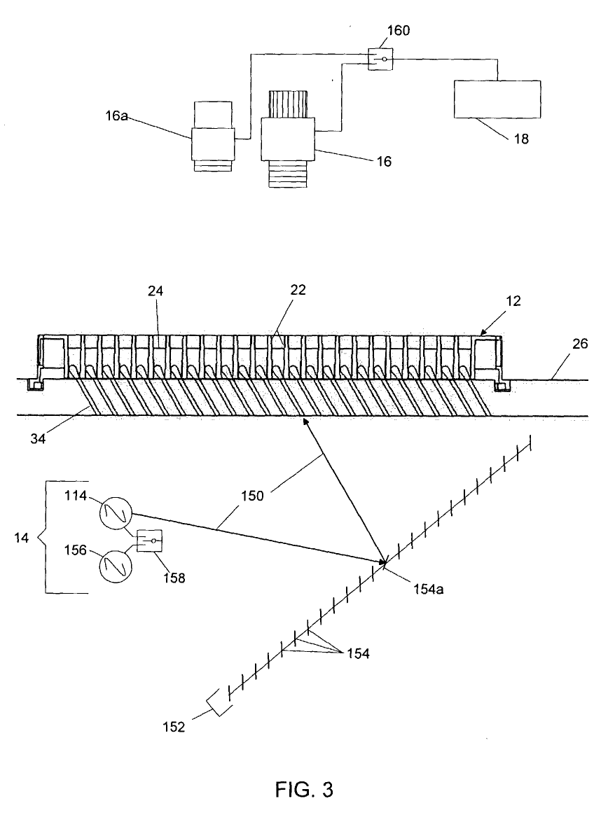

FIG. 3 illustrates a cross sectional view, corresponding with the view of FIG. 2, of an alternative embodiment of the apparatus 10, wherein light source 14 can include a single source of light, 114, though multiple light sources, or arrays of light sources, may also be used. In the embodiment of FIG. 3, light from source 114 can be selectively directed to a sample well 22, as indicated by light path 150, by optical directing means 152, which can utilize reflection, refraction, diffraction and/or other known light directing methods. For example, known micro-electromechanical (MEMS) devices can control the movements of an array of directing optics, e.g., micro-mirrors 154, such that light from a source, such as source 114, can be directed to a desired location, such as a selected sample well 22. It can also be understood that MEMS devices can control the operation of the shutters 36 in the exemplary embodiment of FIG. 2.

In the illustrative embodiment of FIG. 3, mirror 154a can be seen to be rotated with respect to other mirrors 154, such that light from source 114 can follow path 150 into the selected one of sample wells 22. Alternatively, the device 152 can be configured to direct scattered light from a selected sample well 22 to the detector 16. Other means for effecting such re-direction include mirrors, beam-splitters, fiber optics, lenses, etc. In another example embodiment, a single laser can be split into multiple beams to illuminate multiple samples directly.

In one embodiment, light source 14 (or alternatively 114) emanates white light so as to capture all possible phenomena of scattering. In another embodiment, a monochrome light source may be used, or a monochomator, such as a colored filter, can be placed between the light source and the samples, or between the samples and the detector 16, as indicated by filter 40 in FIG. 1. It can be understood that other filter types, including polarizing filters, may also be used. In a further exemplary embodiment, light source 14 may include a luminescent source, and/or a UV source, as indicated at 156 in FIG. 3. Light source 156 can provide light of a defined wavelength such that fluorescence emissions from illuminated samples can be measured, as described previously. It can be understood that light source 156 may be spatially separate from light source 114. For example, light source 156 may be located on the opposite side of tray 12 from light source 114.

A switch 158 can permit switching between the sources 14 (114) and 156, such that detector 16 can measure both the extent of unfolding and the extent of protein aggregation. Where a single detector is used to measure both light scattering and fluorescence, a filter, such as filter 40 in FIG. 1, can be controlled by switch 158, or other switching means, so as to move into and out of the optical path. In one embodiment, a second detector 16a, as shown in FIG. 3, e.g., a fluorescent light detector, can be provided, such that one detector can measure the extent of protein aggregation and the other can measure the extent of unfolding of the biological molecule, with a switch 160 controlling which detector is operating. The switching between the two light sources and/or between the two detectors can be such that the extent of unfolding and the extent of aggregation can be determined essentially simultaneously. For example, switching can be done at every second, every tenth of a second, every one hundredth of a second, or less.

It can be understood that means for distributing samples to the sample wells can be provided. For example, apparatus 10 may be included in a robot or working station having robotic arms to manipulate the samples. Apparatus 10 can be provided in a kit form that can be easily adapted to such existing equipment.

In another embodiment, the invention provides kits containing one or more elements or apparatus necessary for the methods of the invention.

The present invention is further illustrated by the following examples, which should not be construed as limiting in any way. The contents of all cited references including literature references, issued patents, published and non published patent applications as cited throughout this application are hereby expressly incorporated by reference.

The practice of the present invention will employ, unless otherwise indicated, conventional techniques of cell biology, cell culture, molecular biology, transgenic biology, microbiology, recombinant DNA, and immunology, which are within the skill of the art. Such techniques are explained fully in the literature. (See, for example, Molecular Cloning A Laboratory Manual, 2nd Ed., ed. by Sambrook, Fritsch and Maniatis (Cold Spring Harbor Laboratory Press: 1989); DNA Cloning, Volumes I and II (D. N. Glover ed., 1985); Oligonucleotide Synthesis (M. J. Gait ed., 1984); Mullis et al. U.S. Pat. No. 4,683,195; Nucleic Acid Hybridization (B. D. Hames & S. J. Higgins eds. 1984); Transcription And Translation (B. D. Hames & S. J. Higgins eds. 1984); (R. I. Freshney, Alan R. Liss, Inc., 1987); Immobilized Cells And Enzymes (IRL Press, 1986); B. Perbal, A Practical Guide To Molecular Cloning (1984); the treatise, Methods In Enzymology (Academic Press, Inc., N.Y.); Gene Transfer Vectors For Mammalian Cells (J. H. Miller and M. P. Calos eds., 1987, Cold Spring Harbor Laboratory);, Vols. 154 and 155 (Wu et al. eds.), Immunochemical Methods In Cell And Molecular Biology (Mayer and Walker, eds., Academic Press, London, 1987); Handbook Of Experimental Immunology, Volumes I-IV (D. M. Weir and C. C. Blackwell, eds., 1986) (Cold Spring Harbor Laboratory Press, Cold Spring Harbor, N.Y., 1986).

### EXAMPLES

### Example 1

Measure of the Characteristics of Unfolding and Aggregation of a Protein

Multiple identical protein solutions were arranged in two-dimensional arrays of reservoirs which were incubated at different temperatures. These samples were alternatively illuminated with UV light and monochromatic red light. The fluorescence emitted and the light scattered at 90° were measured from the top of the arrangement. The fluorescence emitted and the light scattered intensity data were collected at intervals of time of incubation for all the temperatures simultaneously.

Plots of fluorescence and light scattered at 90° at a given time versus temperature were automatically built for all the samples, and the temperatures of unfolding Tₘ and aggregation T_{agg} were obtained from the fitted sigmoid portion of the curves.

The results are shown in FIGS. 5A and B. The red symbols represent the best fit using a sigmoid Boltzman function.

In another example, the protein was incubated in 5% ethanol; 10% glycerol, NAD⁺ or a control solution and the extent of unfolding was measured at 55° C. A plots of fluorescence at the given temperature versus time was automatically built for all the samples, and the rate of unfolding kᵤ was obtained from the fitted exponential grow portion of the curves for the proteins jumped to 55° C.

The results are shown in FIG. 6. These results show that NAD⁺ has the strongest stabilizing effect on the protein. Glycerol has also a stabilizing effect. On the contrary, ethanol has a destabilizing effect or no effect at all on the protein.

Thus, these results show the possibility of simultaneous detection of the extent of unfolding and the extent of aggregation of a protein. Such methods can be used for high throughput screening of stabilizing conditions and the identification of compounds, e.g., ligands that bind to particular biological molecules.

### Equivalents

The present invention provides among other things novel methods and apparatus for characterizing the stability of biological molecules. While specific embodiments of the subject invention have been discussed, the above specification is illustrative and not restrictive. Many variations of the invention will become apparent to those skilled in the art upon review of this specification. The full scope of the invention should be determined by reference to the claims, along with their full scope of equivalents, and the specification, along with such variations.

Unless otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in this specification and attached claims are approximations that may vary depending upon the desired properties sought to be obtained by the present invention.

All publications and patents mentioned herein are hereby incorporated by reference in their entirety as if each individual publication or patent was specifically and individually indicated to be incorporated by reference. In case of conflict, the present application, including any definitions herein, will control.

1. A method for characterizing aggregation of a plurality of biological samples, comprising:
a) providing a plurality of biological samples, wherein each composition comprises at least one biological molecule;
b) exposing the plurality of biological samples to one or more light sources; and
c) determining the amount of light scattered by said plurality of biological samples upon exposure to said one or more light sources, thereby characterizing aggregation of said biological samples.

2. The method of claim 1, wherein the scattered light is due to Mie scattering.

3. The method of claim 1, wherein the light source is one or more lasers.

4. The method of claim 1, wherein the light source is one or more non-laser lights.

5. The method of claim 4, wherein the non-laser light is one or more of the following: a light emitting diode (LED), a white light source, a monochromatic light source, an incandescent light source, a Xenon-arc lamp, a tungsten-halogen lamp, an ultraviolet light source, a luminescent light source, and a low intensity light source having an intensity in a range of 1.5 to 2.0 μW/mm².

6. The method of claim 4, wherein the non-laser light is a plurality of light emitting diodes (LEDs).

7. The method of claim 1, wherein determining the amount of light scattered comprises detecting the amount of non-scattered light.

8. The method of claim 1, wherein determining the amount of light scattered comprises detecting the amount of scattered light.

9. The method of claim 1, which further comprises detecting the angle of the light scattered.

10. The method of claim 1, which further comprises passing the light source through an optical filter before exposure to the plurality of biological samples.

11. The method of claim 10, wherein the optical filter is a monochromator.

12. The method of claim 10, wherein the optical filter is a polarizing filter.

13. The method of claim 1, wherein said plurality of biological samples comprises at least 5 biological samples.

14. The method of claim 1, wherein said plurality of biological samples comprises at least 10 biological samples.

15. The method of claim 1, wherein said plurality of biological samples comprises at least 15 biological samples.

16. The method of claim 1, wherein said plurality of biological samples comprises at least 20 biological samples.

17. The method of claim 1, wherein said plurality of biological samples comprises at least 50 biological samples.

18. The method of claim 1, wherein said plurality of biological samples comprises at least 96 biological samples.

19. The method of claim 1, wherein said plurality of biological samples comprises at least 250 biological samples.

20. The method of claim 1, wherein said plurality of biological samples comprises at least 384 biological samples.

21. The method of claim 1, wherein said plurality of biological samples comprises at least 1000 biological samples.

22. The method of claim 1, wherein said plurality of biological samples comprises at least 1536 biological samples.

23. The method of claim 1, wherein the plurality of biological samples comprise at least one polypeptide.

24. The method of claim 1, which further comprises determining the aggregation rate (k_{agg}) of said one or more biological samples.

25. The method of claim 1, wherein the plurality of biological samples are contained in a plurality of wells of a microtiter plate.

26. The method of claim 1, comprising preparing the plurality of compositions in an automated fashion.

27. The method of claim 1, wherein characterizing aggregation of said plurality of biological samples is determined as a function of time.

28. The method of claim 1, wherein characterizing aggregation comprises determining one or more of the following: the aggregation state of the biological sample, the aggregation kinetics of the biological sample, or the aggregation dynamics of the biological sample.

29. The method of claim 1, wherein said plurality of biological samples comprises at least one biological molecule in a plurality of test conditions.

30. The method of claim 1, wherein said plurality of biological samples comprises at least one mixture of biological molecules in a plurality of test conditions.

31. The method of claim 1, wherein said plurality of biological samples comprises a plurality of biological molecule in one or more test conditions.

32. The method of claim 31, wherein said plurality of biological samples comprises a plurality of biological molecule in a plurality of test conditions.

33. The method of claim 1, which further comprises comparing a property of aggregation of at least one biological sample in at least one test condition to a property of aggregation of said biological sample in a reference condition.

34. The method of claim 33, wherein a property of aggregation of at least one biological sample is determined in at least 2 test conditions.

35. The method of claim 33, wherein a property of aggregation of at least one biological sample is determined in at least 5 test conditions.

36. The method of claim 33, wherein a property of aggregation of at least one biological sample is determined in at least 10 test conditions.

37. The method of claim 33, wherein a property of aggregation of at least one biological sample is determined in at least 20 test conditions.

38. The method of claim 33, wherein a property of aggregation of at least one biological sample is determined in at least 50 test conditions.

39. The method of claim 33, wherein a property of aggregation of at least one biological sample is determined in at least 100 test conditions.

40. The method of claim 33, wherein said test conditions differ from said reference condition in one or more of the following: a biochemical condition, pressure, electric current, time, concentration of the biological molecule, and presence of a test compound.

41. The method of claim 33, wherein said biochemical condition is one or more of the following: pH, ionic strength, salt concentration, oxidizing agent, reducing agent, detergent, glycerol, metal ions, salt, cofactor concentration, ligand concentration, and coenzyme concentration.

42. The method of claim 33, wherein at least one test condition comprises the presence of one or more potential ligands of a biological molecule in said biological sample.

43. The method of claim 42, wherein a change in a property of aggregation of said biological sample in the presence of a potential ligand relative to the property of aggregation of said biological sample in the absence of the potential ligand indicates that the potential ligand interacts with a biological molecule in said biological sample.

44. The method of claims 1 or 33, which further comprises bringing the temperature of said plurality of biological samples to one or more end temperatures before determining the amount of light scattered.

45. The method of claim 44, wherein said one or more end temperatures are lower than the aggregation temperatures of said plurality of biological samples in a reference condition.

46. The method of claim 45, wherein said one or more end temperatures are lower than the aggregation temperatures of said plurality of biological samples in a reference condition by at least 5° C.

47. The method of claim 45, wherein said one or more end temperatures are lower than the aggregation temperatures of said plurality of biological samples in a reference condition by less than 5° C.

48. The method of claim 45, wherein characterizing aggregation of at least one biological sample is determined at one or more end temperatures as a function of time.

49. The method of claim 45, wherein characterizing aggregation of said plurality of biological samples is determined over a range of end temperatures.

50. The method of claim 49, wherein characterizing aggregation of at least one biological sample is determined over a range of end temperatures by essentially simultaneously bringing a plurality of biological samples comprising a biological molecule to a plurality of end temperatures.

51. The method of claim 49, wherein characterizing aggregation of at least one biological sample is determined over a range of end temperatures by sequentially bringing a biological sample to a plurality of end temperatures.

52. The method of claim 51, wherein the range of end temperatures is sequentially increased.

53. The method of claim 44, wherein characterizing aggregation of at least one biological sample is determined at 2 or more end temperatures.

54. The method of claim 53, wherein characterizing aggregation of at least one biological sample is determined at 5 or more end temperatures.

55. The method of claim 53, wherein characterizing aggregation of at least one biological sample is determined at 10 or more end temperatures.

56. The method of claim 53, wherein characterizing aggregation of at least one biological sample is determined at 20 or more end temperatures.

57. The method of claim 1, which further comprises exposing said plurality of biological samples to a temperature gradient and characterizing aggregation of said plurality of biological samples as a function of temperature.

58. The method of claim 1, which further comprises determining the extent of unfolding of said one or more biological molecules in said plurality of biological samples.

59. The method of claim 58, wherein the extent of unfolding of said one or more biological molecules in said plurality of biological samples is determined by fluorescence emission, circular dichroism, or differential scanning calorimetry.

60. The method of claim 59, wherein the extent of unfolding of said one or more biological molecules is determined using a fluorophore selected from the group consisting of: thioinosine, N-ethenoadenosine, formycin, dansyl derivatives, fluorescein derivatives, 6-propionyl-2-(dimethylamino)-napthalene (PRODAN), 2-anilinonapthalene, N-arylamino-naphthalene sulfonate derivatives, 1-anilinonaphthalene-8-sulfonate (1,8-ANS), 2-anilinonaphthalene-6-sulfonate (2,6-ANS), 2-aminonaphthalene-6-sulfonate, N,N-dimethyl-2-aminonaphthalene-6-sulfonate, N-phenyl-2-aminonaphthalene, N-cyclohexyl-2-aminonaphthalene-6-sulfonate, N-phenyl-2-aminonaphthalene-6-sulfonate, N-phenyl-N-methyl-2-aminonaph-thalene-6-sulfonate, N-(o-toluyl)-2-aminonaphthalene-6-sulfonate, N-(m-toluyl)-2-aminonaphthalene-6-sulfonate, N-(p-toluyl)-2-aminonaphthalene-6-sulfonate, 2-(p-toluidinyl)-naphthalene-6-sulfonic acid (2,6-TNS), 4-(dicyanovinyl)julolidine (DCVJ), 6-dodecanoyl-2-dimethylaminonaphthalene (LAURDAN), 6-hexadecanoyl-2-(((2-(trimethylammonium)ethyl)methyl)amino) naphthalenechl oride (PATMAN), nile red, N-phenyl-1-naphthylamine, 1,1-dicyano-2-[6-(dimethylamino)naphthalen-2-yl]propene (DDNP), 4,4′-dianilino-1,1-binaphthyl-5,5-disulfonic acid (bis-ANS), and DAPOXYL™ derivatives.

61. The method of claim 60, wherein the extent of unfolding of said one or more biological molecules is determined using bis-ANS fluorescence.

62. The method of claim 59, wherein the extent of unfolding of said one or more biological molecules is determined using intrinsic tryptophan fluorescence.

63. The method of claim 58, which further comprises determining the rate of unfolding (kᵤ) and the rate of aggregation (k_{agg}) of said one or more biological molecules.

64. The method of claim 58, which further comprises determining the temperature of unfolding (Tₘ) of said one or more biological molecules.

65. The method of claim 58, wherein said plurality of biological samples are alternatively exposed to a UV light and a light scattering light source.

66. The method of claim 65, wherein the UV light and the light scattering light source are computer controlled to be switched on and off alternatively.

67. A method for characterizing aggregation of a plurality of biological samples, comprising:
a) providing a plurality of biological samples, wherein each composition comprises at least one biological molecule;
b) exposing the plurality of biological samples to one or more light scattering light sources;
c) determining the amount of light scattered by said plurality of biological samples upon exposure to said one or more light scattering light sources;
d) increasing the temperature of said plurality of biological samples in a controlled manner by a pre-determined level; and
e) repeating steps b-d, thereby characterizing aggregation of said biological samples.

68. The method of claim 67, wherein the temperature is increased until a pre-determined end temperature is reached.

69. The method of claim 67, wherein the temperature is increased until no further significant change in the intensities of the light scatted by the compositions is observed.

70. The method of claim 67, wherein said plurality of biological samples comprise at least one biological molecule in a plurality of test conditions.

71. An apparatus for measuring an extent of aggregation in at least one molecular sample, the apparatus comprising:
a light source positioned to illuminate the at least one molecular sample;
a sample container containing the at least one molecular sample;
a light guide positioned in an optical path between the light source and the sample container to direct light from the light source into the at least one molecular sample;
a scattered light detector positioned to determine an amount of light scattered from the at least one molecular sample, the scattered light detector producing a signal proportional to the amount of light; and
a processor in communication with the scattered light detector to receive and process the signal from the light detector to determine the extent of aggregation in the at least one molecular sample.

72. The apparatus of claim 71, wherein the scattered light arises from Mie scattering from the at least one molecular sample illuminated by the light source.

73. The apparatus of claim 71, wherein the light guide directs light into the at least one molecular sample at an angle with respect to the optical path between the at least one molecular sample and the detector corresponding to an enhanced scattering direction, such that the detector captures scattered light without capturing incident illumination.

74. The apparatus of claim 73, wherein the angle is less that 45°.

75. The apparatus of claim 74, wherein the angle is in a range from 15° to 30°.

76. The apparatus of claim 71, comprising a luminescence detector positioned to receive fluorescence emanating from the at least one molecular sample, the luminescence detector producing a signal proportional to the received fluorescence, the processor receiving and processing the signal from the luminescence detector to determine an extent of unfolding in the molecular sample.

77. The apparatus of claim 76, comprising a switch to select between the processor receiving the signal from the scattered light detector and the processor receiving the signal from the luminescence detector.

78. The apparatus of claim 76, wherein the scattered light detector and the luminscence detector are chosen from a listing of detectors including a photomultiplier, a charged-couple device (CCD) and a CMOS vision sensor.

79. The apparatus of claim 71, further comprising a luminescent light source to illuminate the at least one molecular sample, the detector receiving fluorescence emanating from the at least one molecular sample resulting from the illumination by the luminescent light source and producing a signal proportional to the received fluorescence, the processor receiving and processing the signal from the detector to determine an extent of unfolding in the molecular sample.

80. The apparatus of claim 79, comprising a switch to selectively operate the luminescent light source.

81. The apparatus of claim 79, comprising a switch to selectively toggle an optical filter such that the detector alternates between receiving the fluorescence and receiving the scattered light.

82. The apparatus of claim 79, wherein the detector is chosen from a listing of detectors including a photomultiplier, a charged-couple device (CCD) and a CMOS vision sensor.

83. The apparatus of claim 71, wherein the sample container includes an array of sample wells, each sample well being sized to contain one of the at least one molecular samples.

84. The apparatus of claim 83, wherein at least one sample well is spatially separated from another sample well to inhibit cross-contamination of contents of the sample wells.

85. The apparatus of claim 83, wherein at least one sample well is optically isolated from another sample well to inhibit scattered light from the molecular sample in the at least one sample well from illuminating the molecular sample in the other sample well.

86. The apparatus of claim 83, wherein the light guide comprises a collimator positioned in the optical path between the light source and the sample container, the collimator substantially collimating light from the light source into the sample wells.

87. The apparatus of claim 86, wherein the collimator is an array of optical fibers.

88. The apparatus of claim 87, wherein at least some of the optical fibers are each optically aligned with a respective sample well within the sample container.

89. The apparatus of claim 83, further comprising means for selectively directing light from the light source to at least one of the sample wells.

90. The apparatus of claim 83, further comprising means for selectively occluding the optical path between the light source and at least one of the sample wells.

91. The apparatus of claim 83, further comprising means for selectively directing scattered light from at least one of the sample wells to the scattered light detector.

92. The apparatus of claim 83, further comprising means for selectively occluding light from at least one of the sample wells to the scattered light detector.

93. The apparatus of claim 83, further comprising a heating element for heating the sample container.

94. The apparatus of claim 93, wherein the heating element is configured to create a temperature gradient across the sample container.

95. The apparatus of claim 93, wherein the heating element is configured to selectively heat at least one selected sample well, such that the at least one selected sample well is heated to a temperature distinct from other sample wells.

96. The apparatus of claim 71, wherein the light source is at least one of a group of light sources including a laser, a light emitting diode (LED), a cluster of LEDs, a white light source, a monochromatic light source, an incandescent light source, a Xenon-arc lamp, a tungsten-halogen lamp, an ultraviolet light source and a luminescent light source.

97. The apparatus of claim 71, wherein the light source is at least one of a non-coherent and a low-intensity light source.

98. The apparatus if claim 97, wherein the light source intensity is in a range of 1.5 to 2.0 μW/mm².

99. The apparatus of claim 71, further comprising an optical filter positioned in the optical path between the light source and the at least one molecular sample to illuminate the at least one molecular sample with monochromatic light.

100. The apparatus of claim 99, wherein the optical filter is a monochromator.

101. The apparatus of claim 99, wherein the optical filter is a polarizing filter.

102. The apparatus of claim 71, wherein the scattered light detector is chosen from a listing of detectors including a photomultiplier, a charged-couple device (CCD) and a CMOS vision sensor.

103. The apparatus of claim 71, further comprising a heating element for heating the sample container.

104. The apparatus of claim 71, wherein the scattered light detector detects the light scattered from the at least one molecular sample.

105. The apparatus of claim 104, wherein the scattered light detector detects the angle of the scattered light.

106. The apparatus of claim 71, wherein the scattered light detector detects non-scattered light.

107. An apparatus for measuring an extent of aggregation in a plurality of molecular samples, the apparatus comprising:
a sample container containing the molecular samples;
a light source positioned to illuminate selected ones of the molecular samples;
a scattered light detector positioned to determine an amount of light scattered from the selected ones of the molecular samples, the scattered light detector producing a signal proportional to the amount of light, and
a processor in communication with the scattered light detector to receive and process the signal from the scattered light detector to determine the extent of aggregation in the selected ones of the molecular samples.

108. The apparatus of 107, wherein the scattered light results from Mie scattering.

109. The apparatus of claim 107, comprising a collimator positioned in an optical path between the light source and the sample container, the collimator substantially collimating light from the light source into the molecular samples.

110. The apparatus of claim 109, wherein the collimator is an array of optical fibers.

111. The apparatus of claim 110, wherein at least some of the optical fibers are each optically aligned with a respective molecular sample within the sample container.

112. The apparatus of claim 109, wherein the collimator is positioned at an angle with respect to an optical path between the molecular samples and the detector.

113. The apparatus of claim 112, wherein the angle is less that 45°.

114. The apparatus of claim 113, wherein the angle is in a range from 15° to 30°.

115. The apparatus of claim 114, wherein the sample container includes an array of sample wells, each sample well being sized to contain one of the molecular samples and each sample well being optically isolated from other sample wells of the array to inhibit scattered light from the molecular sample in the sample well from illuminating the molecular sample in the other sample wells.

116. The apparatus of claim 107, further comprising optical directing means for selectively directing light from the light source to at least one of the molecular samples.

117. The apparatus of claim 116, wherein the optical directing means comprises micro-electromechanical devices selectively controlling movements of an array of directing optics to form an optical path between the light source and the at least one molecular sample.

118. The apparatus of claim 107, further comprising means for selectively occluding the optical path between the light source and at least one of the molecular samples.

119. The apparatus of claim 107, further comprising means for selectively directing scattered light from at least one of the molecular samples to the scattered light detector.

120. The apparatus of claim 107, further comprising means for selectively occluding light from at least one of the molecular samples to the scattered light detector.

121. The apparatus of claim 107, wherein the light source is at least one of a group of light sources including a laser, a light emitting diode (LED), a cluster of LEDs, a white light source, a monochromatic light source, an incandescent light source, a Xenon-arc lamp, a tungsten-halogen lamp, an ultraviolet light source and a luminescent light source.

122. The apparatus of claim 107, wherein the light source is a low intensity light source.

123. The apparatus if claim 122, wherein the light source intensity is in a range of 1.5 to 2.0 μW/mm².

124. The apparatus of claim 107, wherein the scattered light detector detects the light scattered from the at least one molecular sample.

125. The apparatus of claim 124, wherein the scattered light detector detects the angle of the scattered light.

126. The apparatus of claim 107, wherein the scattered light detector detects non-scattered light.

127. An apparatus for measuring at least one of an extent of aggregation in a plurality of molecular samples and an extent of unfolding in a plurality of molecular samples, the apparatus comprising:
an array of sample wells, each sample well being sized to contain one of the molecular samples;
a light source positioned to illuminate selected ones of the sample wells;
a light guide positioned in an optical path between the light source and the sample container to direct light from the light source into the sample wells;
a light detector positioned to receive at least one of scattered light and fluorescence from the molecular samples in the selected ones of the sample wells, the light detector producing a signal proportional to the received light; and
a processor in communication with the light detector to receive and process the signal from the light detector to determine the extent of aggregation in the molecular samples in the selected ones of the sample wells when the received light is scattered light and to determine the extent of unfolding in the molecular samples in the selected ones of the sample wells when the received light is fluorescence.

128. The apparatus of claim 127, wherein the light source comprises at least one of a low intensity light source and a luminescent light source, light from the low intensity light source passing through the selected ones of the sample wells and scattered by the molecular sample being received as scattered light at the detector, and the detector receiving fluorescence emanating from the molecular samples in the selected ones of the sample wells illuminated by the luminescent light source.

129. The apparatus of claim 128, comprising a switch to selectively operate the low intensity light source and the luminescent light source.

130. The apparatus of claim 127, wherein the detector comprises a scattered light detector and a fluorescence detector, the scattered light detector receiving light from the light source passing through the selected ones of the sample wells and scattered by the molecular samples in the selected ones of the sample wells, the fluorescence detector receiving fluorescence emanating from the molecular samples in the selected ones of the sample wells illuminated by the light source.

131. The apparatus of claim 130, comprising a switch to select between the processor receiving the signal from the scattered light detector and the processor receiving the signal from the fluorescence detector.

132. An apparatus for measuring at least one of an extent of aggregation in a plurality of molecular samples and an extent of unfolding in a plurality of molecular samples, the apparatus comprising:
an array of sample wells, each sample well being sized to contain one of the molecular samples;
a first light source positioned to illuminate first selected ones of the sample wells;
a second light source positioned to illuminate second selected ones of the sample wells;
a light guide positioned in an optical path between the light sources and the sample container to direct light from the light sources into the sample wells;
a light detector positioned to receive at least one of light from the first light source passing through the first selected ones of the sample wells and scattered by the molecular sample and fluorescence emanating from the molecular samples in the second selected ones of the sample wells being illuminated by the second light source, the light detector producing a signal proportional to the received light; and
a processor in communication with the light detector to receive and process the signal from the light detector to determine the extent of aggregation in the molecular samples in the first selected ones of the sample wells when the received light is scattered light and to determine the extent of unfolding in the molecular samples in the second selected ones of the sample wells when the received light is fluorescence.

133. The apparatus of claim 132, wherein the first light source comprises a low intensity light source.

134. The apparatus of claim 132, comprising a switch to selectively operate the first light source and the second light source.

135. The apparatus of claim 132, wherein the detector comprises a scattered light detector and a fluorescence detector, the scattered light detector receiving the scattered light from the first selected ones of the sample wells, the fluorescence detector receiving the fluorescence emanating from the molecular samples in the second selected ones of the sample wells.

136. The apparatus of claim 135, comprising a switch to select between the processor receiving the signal from the scattered light detector and the processor receiving the signal from the fluorescence detector.

137. An apparatus for measuring light scattered by a plurality of molecular samples, comprising:
at least one light source to illuminate the samples to provide scattered light due to Mie scattering; and
a detector to detect the scattered light from all samples simultaneously, wherein
the samples are illuminated such that the amount of scattered light detected is optimized without detecting incident light.

138. The apparatus in claim 137, wherein the light source is pre-selected such that a smallest dimension of particles expected to scatter light exceeds a wavelength of light from the light source.

139. The apparatus in claim 137, further comprising means to direct light from the light source to the samples such that an angle between incident illumination and an axis of optical detection is less than 45°.

140. The apparatus in claim 139, wherein the angle between incident illumination and the axis of optical detection is between 15° and 30°.

141. The apparatus of claim 139, wherein the means to direct light comprises relative positioning of the light source and detector.

142. The apparatus of claim 139, wherein the means to direct light comprises a light guide.

143. The apparatus of claim 142, wherein the light guide comprises a plurality of individual light guides directing light to respective ones of the plurality of samples.

144. The apparatus of 137, further comprising means to accommodate changing pluralities of samples, while selectively illuminating the samples, maintaining the at least one light source and inhibiting crosstalk between the samples.

145. The apparatus of 144, wherein the means to accommodate changing pluralities of samples is a light guide.

146. The apparatus of claim 137, wherein the at least one light source is at least one of low intensity and non-coherent.
